(19) United States
(12) Patent Application Publication
(10) Pub. No.: US 2005/0220768 A1
(43) Pub. Date: Oct. 6, 2005

(54) Materials and methods for treating ocular-related disorders

(75) Inventors: Duncan L. McVey, Derwood, MD (US); Douglas E. Brough, Gaithersburg, MD (US); Imre Kovesdi, Rockville, MD (US); Lisa Wei, Gaithersburg, MD (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: GenVec, Inc., Gaithersburg, MD (US)

(21) Appl. No.: 11/138,931

(22) Filed: May 26, 2005

Related U.S. Application Data
(63) Continuation of application No. PCT/US03/38169, filed on Dec. 1, 2003.
(60) Provisional application No. 60/430,617, filed on Dec. 2, 2002.

(51) Int. Cl. A61K 48/00

(52) U.S. Cl. 424/93.2; 514/44

(57) ABSTRACT

The invention is directed to a method of delivering a gene product to an animal. The method comprises administering an expression vector comprising a nucleic acid sequence operably linked to a promoter and encoding a gene product, and upregulating transcription of the nucleic acid sequence in the ocular cell. The expression vector can be an adenoviral vector. The invention further provides a method of prophylactically or therapeutically treating an animal for at least one ocular-related disorder. The method comprises contacting an ocular cell with an expression vector comprising a nucleic acid sequence encoding an inhibitor of angiogenesis and/or a neurotrophic agent. In one aspect, the method further comprises upregulating transcription of the nucleic acid sequence. Preferably, if 2×10⁸ adenoviral particles of the inventive method are administered to a mouse, the level of expression of the nucleic acid sequence is not diminished more than ten-fold at 28 days post-administration.

### CROSS-REFERENCE TO RELATED APPLICATIONS

This patent application is a continuation of copending International Patent Application No. PCT/US03/38169, filed Dec. 1, 2003, which claims the benefit of U.S. Provisional Patent Application No. 60/430,617, filed Dec. 2, 2002.

### FIELD OF THE INVENTION

The invention relates to a method of prophylactically or therapeutically treating an ocular disorder as well as materials useful for treating an ocular disorder.

### BACKGROUND OF THE INVENTION

An overwhelming majority of the world's population will experience some degree of vision loss in their lifetime. Vision loss affects virtually all people regardless of age, race, economic or social status, or geographical location. Ocular-related disorders, while often not life threatening, necessitate life-style changes that jeopardize the independence of the afflicted individual. Vision impairment can result from most all ocular disorders, including diabetic retinopathies, proliferative retinopathies, retinal detachment, toxic retinopathies, retinal vascular diseases, retinal degenerations, vascular anomalies, age-related macular degeneration and other acquired disorders, infectious diseases, inflammatory diseases, ocular ischemia, pregnancy-related disorders, retinal tumors, choroidal tumors, choroidal disorders, vitreous disorders, trauma, cataract complications, dry eye, and inflammatory optic neuropathies.

Leading causes of severe vision loss and blindness are ocular-related disorders wherein the vasculature of the eye is damaged or insufficiently regulated. Ocular-related diseases comprising a neovascularization aspect are many and include, for example, exudative age-related macular degeneration, diabetic retinopathy, corneal neovascularization, choroidal neovascularization, neovascular glaucoma, cyclitis, Hippel-Lindau Disease, retinopathy of prematurity, pterygium, histoplasmosis, iris neovascularization, macular edema, glaucoma-associated neovascularization, and the like. It is likely that severe vision loss does not result directly from neovascularization, but the effect of neovascularization on the retina. The retina is a delicate ocular membrane on which images are received. Near the center of the retina is the macula lutea, an oval area of retinal tissue where visual sense is most acute. The retina is most delicate at the fovea centralis, the central depression located in the center of the macula. Damage of the retina, i.e., retinal detachment, retinal tears, or retinal degeneration, is directly connected to vision loss. While a common cause of retinal detachment, retinal tears, and retinal degeneration is abnormal, i.e., uncontrolled, vascularization of various ocular tissues, this is not always the case. Atrophic complications associated with age-related macular degeneration, nonproliferative diabetic retinopathy, and inflammatory ocular damage are not associated with neovascularization, but can result in severe vision loss if not treated. Disorders associated with both neovascular and atrophic components, such as age-related macular degeneration and diabetic retinopathy, are particularly difficult to treat due to the emergence of a wide variety of complications.

Age-related macular degeneration is the leading cause of blindness in patients over 65 years of age. As the elderly population of the world increases, the incidence of age-related macular degeneration is expected to increase dramatically, reaching a predicted 7.5 million cases in the United States alone by the year 2030 (Hyman et al., Am. J Epidemiol., 118, 213-227 (1983)). Age-related macular degeneration (AMD) is a progressive, degenerative disorder of the eye resulting initially in loss of visual acuity. Complications arising with advanced age-related macular degeneration include atrophic and exudative complications. Atrophic complications stem from retinal pigment epithelial cell loss resulting in atrophy of the retinal pigment epithelium (RPE). Exudative complications include disciform scars (i.e., scarring involving fibrous elements) and neovascularization. Severe vision loss occurs as neovascularization or atrophy disturbs the foveal center (Bressler et al., Ophthalmology, 102, 1206-1211 (1995)). Ultimately, blindness from age-related macular degeneration stems from degeneration of the RPE and the subsequent death of photoreceptors.

Similarly, diabetic retinopathy is subdivided into a nonproliferative stage, which typically occurs first, and a proliferative stage. Vision loss associated with nonproliferative diabetic retinopathy stems from retinal edema, in particular diabetic macular edema, resulting from vascular leakage. Focal and diffuse vascular leakage occurs as a result of microvascular abnormalities, intraretinal microaneurysms, capillary closure, and retinal hemorrhages. Prolonged periods of vascular leakage ultimately lead to thickening of the basement membrane and formation of soft and hard exudates. Nonproliferative diabetic retinopathy is also characterized by loss of retinal pericytes. The proliferative stage of diabetic retinopathy is characterized by neovascularization and fibrovascular growth (i.e., scarring involving glial and fibrous elements) from the retina or optic nerve over the inner surface of the retina or disc or into the vitreous cavity. Retinal neovascularization is the leading cause of vision loss associated with proliferative diabetic retinopathy.

For many ocular-related disorders, no efficient therapeutic options currently are available. Laser photocoagulation involves administering laser burns to various areas of the eye and is used in the treatment of many neovascularization-linked disorders. For example, focal macular photocoagulation is used to treat areas of vascular leakage outside the macula (Murphy, Amer. Family Physician, 51(4), 785-796 (1995)). Similarly, neovascularization, in particular, advanced proliferative retinopathy, is commonly treated with scatter or panretinal photocoagulation. However, laser treatment may cause permanent blind spots corresponding to the treated areas. Laser treatment may also cause persistent or recurrent hemorrhage, increase the risk of retinal detachment, or induce neovascularization or fibrosis. With respect to age-related macular degeneration, many patients eventually experience severe vision loss in spite of treatment. Other treatment options for ocular-related disorders include thermotherapy, radiation therapy, surgery, e.g., macular translocation, removal of excess ocular tissue, and the like. However, in most cases, all available treatment options have limited therapeutic effect, require repeated, costly procedures, and/or are associated with dangerous side-effects.

Given the prevalence of ocular-related disorders, there remains a need for an effective prophylactic and therapeutic treatment of ocular-related disorders, in particular those ocular-related disorders associated with both atrophic and neovascular complications, such as diabetic retinopathy and age-related macular degeneration. Accordingly, the invention provides materials and methods for prophylactically and therapeutically treating ocular-related disorders, including treatment of nonproliferative complications and proliferative complications. This and other advantages of the invention will become apparent from the detailed description provided herein.

### BRIEF SUMMARY OF THE INVENTION

The invention provides a method for the prophylactic or therapeutic treatment of ocular-related disorders. In particular, the invention provides a method of prophylactically or therapeutically treating an animal for at least one ocular-related disorder, such as ocular neovascularization and age-related macular degeneration. The method comprises contacting an ocular cell with (a) an expression vector comprising a nucleic acid sequence encoding an inhibitor of angiogenesis and the same or different nucleic acid sequence encoding a neurotrophic agent, or (b) different expression vectors, each comprising a nucleic acid sequence encoding an inhibitor of angiogenesis and/or a neurotrophic agent. The nucleic acid sequence encoding the inhibitor of angiogenesis and/or the nucleic acid sequence encoding the neurotrophic agent is (are) expressed, thereby resulting in the production of the inhibitor of angiogenesis and/or the neurotrophic agent to prophylactically or therapeutically treat the animal for an ocular-related disorder. Preferably, the nucleic acid sequence encoding the inhibitor of angiogenesis and the nucleic acid sequence encoding the neurotrophic agent are the same nucleic acid sequence. More preferably, the nucleic acid sequence encodes a factor comprising both anti-angiogenic and neurotrophic activity. Most preferably, the factor is PEDF.

In addition, the invention provides a viral vector comprising a nucleic acid sequence encoding pigment epithelium-derived factor (PEDF) or a therapeutic fragment thereof. The nucleic acid sequence is operably linked to regulatory sequences necessary for expression of PEDF or a therapeutic fragment thereof. Preferably, the viral vector is an adenoviral vector or an adeno-associated viral vector. Also preferably, the viral vector further comprises one or more additional nucleic acid sequences encoding therapeutic substances other than PEDF or a therapeutic fragment thereof.

In addition, the invention provides a method of delivering a gene product to the eye. The method comprises administering to an eye of an animal a first expression vector comprising a nucleic acid sequence operably linked to a promoter and encoding a gene product, such that the expression vector transduces an ocular cell and the nucleic acid sequence is transcribed. The method further comprises upregulating transcription of the nucleic acid sequence in the ocular cell. Upregulating transcription does not comprise administering a pyrogen, and can comprise exposing the ocular cell to saline, trehalose, a protein, a nucleic acid, a lipid, a steroid derivative, diclofenac sodium and misoprostol, dixlurenac, combretastatin, a protein kinase C (PKC) inhibitor, a tyrosine kinase inhibitor, hyaluronic acid, a second expression vector, a histone deacetylase inhibitor, retinoic acid, cold, light, radiation, microwaves, ultrasound, or physical trauma.

The invention further provides a method of delivering a gene product to a mammal. The method comprises (a) administering to the mammal an adenoviral vector deficient in all replication-essential gene functions of the E4 region of the adenoviral genome and comprising a nucleic acid sequence operably linked to a promoter and encoding a gene product, such that the adenoviral vector transduces a host cell and the nucleic acid sequence is transcribed to produce a gene product. The method further comprises (b) subsequently upregulating transcription of the nucleic acid sequence in the host cell. Upregulating transcription does not comprise administering a pyrogen, an adenoviral vector, or radiation.

Also provided is a method of prophylactically or therapeutically treating an animal for an ocular-related disorder. The method comprises administering to the animal a first expression vector comprising a nucleic acid sequence encoding an inhibitor of angiogenesis and/or a neurotrophic agent such that the expression vector transduces at least one ocular cell and the nucleic acid sequence is transcribed. The method further comprises upregulating transcription of the nucleic acid sequence. Expression of the inhibitor of angiogenesis and/or a neurotrophic agent is thereby upregulated to prophylactically or therapeutically treat the animal for an ocular-related disorder. Alternatively or additionally, the first expression vector is an adenoviral vector comprising a nucleic acid sequence operably linked to a cellular promoter and encoding an inhibitor of angiogenesis and/or a neurotrophic agent. When administered to a mouse at a dose of 2×10⁸ particles, the level of transcription of the nucleic acid sequence is not diminished more than ten-fold at 28 days post-administration of the adenoviral vector compared to the level of transcription of the nucleic acid sequence at one day post-administration of the adenoviral vector.

The invention further provides a method of delivering a gene product to a mammal, wherein the method comprises (a) administering to a mammal (i) an expression vector comprising a first nucleic acid sequence operably linked to a promoter, such that the expression vector transduces a host cell and the first nucleic acid sequence is transcribed to produce a gene product, and (ii) a second nucleic acid sequence operably linked to a promoter and encoding a retinoic acid receptor, such that the second nucleic acid sequence is transcribed in the host cell to produce the retinoic acid receptor. The method further comprises (b) subsequently administering to the mammal a retinoic acid, thereby upregulating transcription of the first nucleic acid sequence in the host cell.

### BRIEF DESCRIPTION OF THE DRAWINGS

FIG. 1 is a graph correlating luciferase activity (RLU/μg protein) to day post-administration of AdL.11D.

FIG. 2 is a graph correlating luciferase activity (RLU/μg protein) to day post-administration of AdL.11D.

FIG. 3 is a graph correlating luciferase activity (RLU/μg protein) produced from transcription of the luciferase gene in AdL.11D and methods of activating a stress response in an ocular cell.

FIG. 4 is a graph correlating luciferase activity (RLU/μg protein) to day post-administration of AdUb.L.11D, AdYY1.L.11D, and AdJEM1.L.11D.

### DETAILED DESCRIPTION OF THE INVENTION

The invention is directed to methods of prophylactically or therapeutically treating an animal, preferably a human, for at least one ocular-related disorder. The invention also provides materials for treating ocular-related disorders. Ocular-related disorders appropriate for treatment using the present inventive materials and methods include, but are not limited to, diabetic retinopathies, proliferative retinopathies, retinopathy of prematurity, retinal vascular diseases, vascular anomalies, age-related macular degeneration and other acquired disorders, endophthalmitis, infectious diseases, inflammatory diseases, AIDS-related disorders, ocular ischemia syndrome, pregnancy-related disorders, peripheral retinal degenerations, retinal degenerations, toxic retinopathies, cataracts, retinal tumors, corneal neovascularization, choroidal tumors, choroidal disorders, choroidal neovascularization, neovascular glaucoma, vitreous disorders, retinal detachment and proliferative vitreoretinopathy, cyclitis, non-penetrating trauma, penetrating trauma, post-cataract complications, Hippel-Lindau Disease, dry eye, inflammatory optic neuropathies, glaucoma, macular edema, pterygium, iris neovascularization, uveitis, pathologic myopia, surgical-induced disorders, and the like.

In particular, the invention provides a method of prophylactically or therapeutically treating an animal for at least one ocular-related disorder, such as ocular neovascularization. The method comprises contacting an ocular cell with an expression vector comprising a nucleic acid sequence encoding at least one inhibitor of angiogenesis and/or at least one neurotrophic agent. Preferably, the method comprises contacting an ocular cell with an expression vector comprising a nucleic acid sequence encoding an inhibitor of angiogenesis and the same or different nucleic acid sequence encoding a neurotrophic agent. Desirably, the nucleic acid sequence encodes at least one inhibitor of angiogenesis and at least one neurotrophic agent. The ocular neovascularization treated by the present inventive method can be neovascularization of the choroid. The choroid is a thin, vascular membrane located under the retina. Abnormal neovascularization of the choroid results from, for example, photocoagulation, anterior ischemic optic neuropathy, Best's disease, choroidal hemangioma, metallic intraocular foreign body, choroidal nonperfusion, choroidal osteomas, choroidal rupture, bacterial endocarditis, choroideremia, chronic retinal detachment, drusen, deposit of metabolic waste material, endogenous Candida endophthalmitis, neovascularization at ora serrata, operating microscope burn, punctate inner choroidopathy, radiation retinopathy, retinal cryoinjury, retinitis pigmentosa, retinochoroidal coloboma, rubella, subretinal fluid drainage, tilted disc syndrome, Taxoplasma retinochoroiditis, tuberculosis, and the like.

Neovascularization of the cornea is also appropriate for treatment by the method of the invention. The cornea is a projecting, transparent section of the fibrous tunic, the outer most layer of the eye. The outermost layer of the cornea contacts the conjunctiva, while the innermost layer comprises the endothelium of the anterior chamber. Corneal neovascularization stems from, for example, ocular injury, surgery, infection, improper wearing of contact lenses, and diseases such as, for example, corneal dystrophies.

Alternatively, the ocular neovascularization is preferably neovascularization of the retina. Retinal neovascularization is an indication associated with numerous ocular diseases and disorders, many of which are named above. Preferably, the neovascularization of the retina treated in accordance with the present inventive method is associated with diabetic retinopathy. Common causes of retinal neovascularization include ischemia, viral infection, and retinal damage. Neovascularization of the retina can lead to macular edema, subretinal discoloration, scarring, hemorrhaging, and the like. Complications associated with retina neovascularization stem from growth, breakage and leakage of newly formed blood vessels. Vision is impaired as blood fills the vitreous cavity and is not efficiently removed. Not only is the passage of light impeded, but an inflammatory response to the excess blood and metabolites can cause further damage to ocular tissue. In addition, the new vessels form fibrous scar tissue, which, over time, will disturb the retina causing retinal tears and detachment.

The invention also provides a method for prophylactically or therapeutically treating an animal for age-related macular degeneration. The method comprises contacting an ocular cell with an expression vector comprising a nucleic acid sequence encoding at least one inhibitor of angiogenesis and/or at least one neurotrophic factor. Desirably, the expression vector comprises a nucleic acid sequence encoding an inhibitor of angiogenesis and a nucleic acid sequence encoding a neurotrophic agent. More desirably, the nucleic acid sequence encoding the inhibitor of angiogenesis and the nucleic acid sequence encoding the neurotrophic agent are the same nucleic acid sequence. Preferably, the age-related macular degeneration is associated with at least one exudative complication. Exudative complications include, for example, disciform scars (i.e., scarring involving fibrous elements) and neovascularization. Alternatively, the age-related macular degeneration is associated with at least one atrophic complication. Atrophic complications include, for instance, the formation of drusen and basal laminar deposits, irregularity of retinal pigmentation, and accumulation of lipofuscin granules.

By “prophylactic” is meant the protection, in whole or in part, against ocular-related disorders, in particular ocular neovascularization or age-related macular degeneration. By “therapeutic” is meant the amelioration of the ocular-related disorder, itself, and the protection, in whole or in part, against further ocular-related disease, in particular ocular neovascularization or age-related macular degeneration. One of ordinary skill in the art will appreciate that any degree of protection from, or amelioration of, an ocular-related disorder is beneficial to a patient. The invention is particularly advantageous in that a therapeutic agent can be directly applied to affected areas without the harmful side effects of presently employed therapies.

The present inventive methods are useful in the treatment of both acute and persistent, progressive ocular-related disorders. For acute ailments, the expression vector comprising a nucleic acid sequence encoding at least one inhibitor of angiogenesis and/or at least one neurotrophic factor can be administered using a single or multiple applications within a short time period. For persistent ocular-related diseases, such as age-related macular degeneration and diabetic retinopathy, numerous applications of the expression vector may be necessary to realize a therapeutic effect.

One of ordinary skill in the art will appreciate that any of a number of expression vectors known in the art are suitable for use in the present inventive methods. Examples of suitable expression vectors include, for instance, plasmids, plasmid-liposome complexes, and viral vectors, e.g., parvoviral-based vectors (i.e., adeno-associated virus (AAV)-based vectors), retroviral vectors, herpes simplex virus (HSV)-based vectors, AAV-adenoviral chimeric vectors, and adenovirus-based vectors. Any of these expression vectors can be prepared using standard recombinant DNA techniques described in, e.g., Sambrook et al., Molecular Cloning, a Laboratory Manual, 2d edition, Cold Spring Harbor Press, Cold Spring Harbor, N.Y. (1989), and Ausubel et al., Current Protocols in Molecular Biology, Greene Publishing Associates and John Wiley & Sons, New York, N.Y. (1994).

Plasmids, genetically engineered circular double-stranded DNA molecules, can be designed to contain an expression cassette for delivery of the nucleic acid sequence encoding at least one inhibitor of angiogenesis and/or at least one neurotrophic factor to an ocular cell. Although plasmids were the first vector described for administration of therapeutic nucleic acids, the level of transfection efficiency is poor compared with other techniques. By complexing the plasmid with liposomes, the efficiency of gene transfer in general is improved. While the liposomes used for plasmid-mediated gene transfer strategies have various compositions, they are typically synthetic cationic lipids. Advantages of plasmid-liposome complexes include their ability to transfer large pieces of DNA encoding a therapeutic nucleic acid and their relatively low immunogenicity.

Plasmids are often used for short-term expression. However, a plasmid construct can be modified to obtain prolonged expression. It has recently been discovered that the inverted terminal repeats (ITR) of parvovirus, in particular adeno-associated virus (AAV), are responsible for the high-level persistent nucleic acid expression often associated with AAV (see, for example, U.S. Pat. No. 6,165,754). Accordingly, the expression vector can be a plasmid comprising native parvovirus ITRs to obtain prolonged and substantial expression of at least one inhibitor of angiogenesis and/or at least one neurotrophic factor. While plasmids are suitable for use in the present inventive methods, preferably the expression vector is a viral vector.

AAV vectors are viral vectors of particular interest for use in gene therapy protocols. AAV is a DNA virus, which is not known to cause human disease. AAV requires co-infection with a helper virus (i.e., an adenovirus or a herpes virus), or expression of helper genes, for efficient replication. AAV vectors used for administration of a therapeutic nucleic acid have approximately 96% of the parental genome deleted, such that only the terminal repeats (ITRs), which contain recognition signals for DNA replication and packaging, remain. This eliminates immunologic or toxic side effects due to expression of viral genes. In addition, delivering the AAV rep protein enables integration of the AAV vector comprising AAV ITRs into a specific region of genome, if desired. Host cells comprising an integrated AAV genome show no change in cell growth or morphology (see, for example, U.S. Pat. No. 4,797,368). Although efficient, the need for helper virus or helper genes can be an obstacle for widespread use of this vector.

Retrovirus is an RNA virus capable of infecting a wide variety of host cells. Upon infection, the retroviral genome integrates into the genome of its host cell and is replicated along with host cell DNA, thereby constantly producing viral RNA and any nucleic acid sequence incorporated into the retroviral genome. When employing pathogenic retroviruses, e.g., human immunodeficiency virus (HIV) or human T-cell lymphotrophic viruses (HTLV), care must be taken in altering the viral genomic to eliminate toxicity. A retroviral vector can additionally be manipulated to render the virus replication-incompetent. As such, retroviral vectors are thought to be particularly useful for stable gene transfer in vivo: Lentiviral vectors, such as HIV-based vectors, are exemplary of retroviral vectors used for gene delivery. Unlike other retroviruses, HIV-based vectors are known to incorporate their passenger genes into non-dividing cells and, therefore, can be of use in treating atrophic forms of ocular-related disease.

HSV-based viral vectors are suitable for use as an expression vector to introduce nucleic acids into ocular cells. The mature HSV virion consists of an enveloped icosahedral capsid with a viral genome consisting of a linear double-stranded DNA molecule that is 152 kb. Most replication-deficient HSV vectors contain a deletion to remove one or more intermediate-early genes to prevent replication. Advantages of the herpes vector are its ability to enter a latent stage that can result in long-term DNA expression, and its large viral DNA genome that can accommodate exogenous DNA up to 25 kb. Of course, this ability is also a disadvantage in terms of short-term treatment regimens. For a description of HSV-based vectors appropriate for use in the present inventive methods, see, for example, U.S. Pat. Nos. 5,837,532; 5,846,782; 5,849,572; and 5,804,413 and International Patent Applications WO 91/02788, WO 96/04394, WO 98/15637, and WO 99/06583.

Adenovirus (Ad) is a 36 kb double-stranded DNA virus that efficiently transfers DNA in vivo to a variety of different target cell types. For use in the present inventive methods, the virus is preferably made replication deficient by deleting select genes required for viral replication. The expendable E3 region is also frequently deleted to allow additional room for a larger DNA insert. The vector can be produced in high titers and can efficiently transfer DNA to replicating and non-replicating cells. The newly transferred genetic information remains epi-chromosomal, thus eliminating the risks of random insertional mutagenesis and permanent alteration of the genotype of the target cell. However, if desired, the integrative properties of AAV can be conferred to adenovirus by constructing an AAV-Ad chimeric vector. For example, the AAV ITRs and nucleic acid encoding the Rep protein incorporated into an adenoviral vector enables the adenoviral vector to integrate into a mammalian cell genome. Therefore, AAV-Ad chimeric vectors are an interesting option for use in the invention.

Preferably, the expression vector of the present inventive methods is a viral vector; more preferably, the expression vector is an adenoviral vector, e.g., a human adenoviral vector. In the context of the invention, the adenoviral vector can be derived from any serotype of adenovirus. Adenoviral stocks that can be employed as a source of adenovirus can be amplified from the adenoviral serotypes 1 through 51, which are currently available from the American Type Culture Collection (ATCC, Manassas, Va.), or from any other serotype of adenovirus available from any other source. For instance, an adenovirus can be of subgroup A (e.g., serotypes 12, 18, and 31), subgroup B (e.g., serotypes 3, 7, 11, 14, 16, 21, 34, and 35), subgroup C (e.g., serotypes 1, 2, 5, and 6), subgroup D (e.g., serotypes 8, 9, 10, 13, 15, 17, 19, 20, 22-30, 32, 33, 36-39, and 42-47), subgroup E (serotype 4), subgroup F (serotypes 40 and 41), or any other adenoviral serotype. Preferably, however, an adenovirus is of serotype 2, 5 or 9. However, non-group C adenoviruses can be used to prepare replication-deficient adenoviral gene transfer vectors for delivery of anti-angiogenic factors and/or neurotrophic factors to ocular cells. Preferred adenoviruses used in the construction of non-group C adenoviral gene transfer vectors include Ad12 (group A), Ad7 and Ad35 (group B), Ad30 and Ad36 (group D), Ad4 (group E), and Ad41(group F). Non-group C adenoviral vectors, methods of producing non-group C adenoviral vectors, and methods of using non-group C adenoviral vectors are disclosed in, for example, U.S. Pat. Nos. 5,801,030; 5,837,511; and 5,849,561 and International Patent Applications WO 97/12986 and WO 98/53087.

The adenoviral vector is preferably deficient in at least one gene function required for viral replication, thereby resulting in a “replication-deficient” adenoviral vector. By “replication-deficient” is meant that the adenoviral vector comprises an adenoviral genome that lacks at least one replication-essential gene function (i.e., such that the adenoviral vector does not replicate in typical host cells, especially those in the human patient that could be infected by the adenoviral vector in the course of treatment in accordance with the invention). A deficiency in a gene, gene function, or gene or genomic region, as used herein, is defined as a deletion of sufficient genetic material of the viral genome to impair or obliterate the function of the gene whose nucleic acid sequence was deleted in whole or in part. Deletion of an entire gene region often is not required for disruption of a replication-essential gene function. However, for the purpose of providing sufficient space in the adenoviral genome for one or more transgenes, removal of a majority of a gene region may be desirable. Replication-essential gene functions are those gene functions that are required for replication (e.g., propagation) and are encoded by, for example, the adenoviral early regions (e.g., the E1, E2, and E4 regions), late regions (e.g., the L1-L5 regions), genes involved in viral packaging (e.g., the IVa2 gene), and virus-associated RNAs (e.g., VA-RNA-1 and/or VA-RNA-2). More preferably, the replication-deficient adenoviral vector comprises an adenoviral genome deficient in at least one replication-essential gene function of one or more regions of the adenoviral genome. In this respect, the adenoviral vector is deficient in at least one essential gene function of the E1 region of the adenoviral genome required for viral replication. In addition to a deficiency in the E1 region, the recombinant adenovirus can also have a mutation in the major late promoter (MLP). The mutation in the MLP can be in any of the MLP control elements such that it alters the responsiveness of the promoter, as discussed in International Patent Application WO 00/00628. More preferably, the vector is deficient in at least one essential gene function of the E1 region and at least part of the E3 region (e.g., an Xba I deletion of the E3 region). With respect to the E1 region, the adenoviral vector can be deficient in at least part of the E1a region and at least part of the E1b region. For example, the adenoviral vector can comprise a deletion of the entire E1 region and part of the E3 region of the adenoviral genome (i.e., nucleotides 355 to 3,511 and 28,593 to 30,470). A singly-deficient adenoviral vector can be deleted of approximately nucleotides 356 to 3,329 and 28,594 to 30,469 (based on the adenovirus serotype 5 genome). Alternatively, the adenoviral vector genome can be deleted of approximately nucleotides 356 to 3,510 and 28,593 to 30,470 (based on the adenovirus serotype 5 genome). The endpoints defining the deleted nucleotide portions can be difficult to precisely determine and typically will not significantly affect the nature of the adenoviral vector, i.e., each of the aforementioned nucleotide numbers can be +/−1, 2, 3, 4, 5, or even 10 or 20 nucleotides.

Preferably, the adenoviral vector is “multiply deficient,” meaning that the adenoviral vector is deficient in one or more essential gene functions required for viral replication in each of two or more regions. For example, the aforementioned E1-deficient or E1-, E3-deficient adenoviral vectors can be further deficient in at least one essential gene of the E4 region. Adenoviral vectors deleted of the entire E4 region can elicit lower host immune responses. When E4-deficient, the adenoviral vector genome can comprise a deletion of, for example, nucleotides 32,826 to 35,561 (based on the adenovirus serotype 5 genome), optionally in addition to deletions in the E1 region (e.g., nucleotides 356 to 3,329 or nucleotides 356 to 3,510) and/or deletions in the E3 region (e.g., nucleotides 28,594 to 30,469 or nucleotides 28,593 to 30,470). The adenoviral vector, when multiply replication-deficient, especially in replication-essential gene functions of the E1 and E4 regions, preferably includes a spacer element to provide viral growth in a complementing cell line similar to that achieved by singly replication-deficient adenoviral vectors, particularly an E1-deficient adenoviral vector.

Alternatively, the adenoviral vector lacks all or part of the E1 region and all or part of the E2 region (e.g., the E2A region). However, adenoviral vectors lacking all or part of the E1 region, all or part of the E2 region, and all or part of the E3 region also are contemplated herein. In one embodiment, the adenoviral vector lacks all or part of the E1 region, all or part of the E2 region, all or part of the E3 region, and all or part of the E4 region. Suitable replication-deficient adenoviral vectors are disclosed in U.S. Pat. Nos. 5,851,806 and 5,994,106 and International Patent Applications WO 95/34671 and WO 97/21826. For example, suitable replication-deficient adenoviral vectors include those with at least a partial deletion of the E1a region, at least a partial deletion of the E1b region, at least a partial deletion of the E2a region, and at least a partial deletion of the E3 region. Alternatively, the replication-deficient adenoviral vector can have at least a partial deletion of the E1 region, at least a partial deletion of the E3 region, and at least a partial deletion of the E4 region. Alternatively or in addition, other regions of the adenoviral genome also can be deleted such as the VAI gene and VAII gene as described in International Patent Application No. PCT/US02/29111. Multiply-deficient viral vectors are particularly useful in that such vectors can accept large inserts of exogenous DNA. Indeed; adenoviral amplicons, an example of a multiply-deficient adenoviral vector which comprises only those genomic sequences required for packaging and replication of the viral genome, can accept inserts of approximately 36 kb.

Therefore, in a preferred embodiment, the expression vector of the present inventive method is a multiply-deficient adenoviral vector lacking all or part of the E1 region, all or part of the E3 region, all or part of the E4 region, and, optionally, all or part of the E2 region. In this regard, it has been observed that an at least E4-deficient adenoviral vector expresses a transgene at high levels for a limited amount of time in vivo and that persistence of expression of a transgene in an at least E4-deficient adenoviral vector can be modulated through the action of a trans-acting factor, such as HSV ICP0, Ad pTP, CMV-IE2, CMV-IE86, HIV tat, HTLV-tax, HBV-X, AAV Rep 78, the cellular factor from the U205 osteosarcoma cell line that functions like HSV ICP0, or the cellular factor in PC12 cells that is induced by nerve growth factor, among others. In view of the above, the multiply deficient adenoviral vector (e.g., the at least E4-deficient adenoviral vector) preferably further comprises a nucleic acid sequence encoding a trans-acting factor that modulates the persistence of expression of the nucleic acid sequence encoding at least one inhibitor of angiogenesis and/or at least one neurotrophic factor. Alternatively, the ocular cell is contacted with a second expression vector comprising a nucleic acid sequence encoding a trans-acting factor that modulates the persistence of expression of the nucleic acid sequence encoding at least one inhibitor of angiogenesis and/or at least one neurotrophic factor. Preferably, the nucleic acid sequence encoding the trans-acting factor does not encode an adenoviral E4 region gene product. Whether expressed from the adenoviral vector or supplied by a second expression vector, preferably, the trans-acting factor is the Herpes simplex infected cell polypeptide 0 (HSV ICP0).

It should be appreciated that the deletion of different regions of the adenoviral vector can alter the immune response of the mammal. In particular, deletion of different regions can reduce the inflammatory response generated by the adenoviral vector. Furthermore, the adenoviral vector's coat protein can be modified so as to decrease the adenoviral vector's ability or inability to be recognized by a neutralizing antibody directed against the wild-type coat protein, as described in International Patent Application WO 98/40509. Such modifications are useful for long-term treatment of persistent ocular disorders.

The adenoviral vector, when multiply replication-deficient, especially in replication-essential gene functions of the E1 and E4 regions, preferably includes a spacer element to provide viral growth in a complementing cell line similar to that achieved by singly replication-deficient adenoviral vectors, particularly an adenoviral vector comprising a deficiency in the E1 region. In the preferred E4-adenoviral vector of the present invention wherein the L5 fiber region is retained, the spacer is desirably located between the L5 fiber region and the right-side ITR. More preferably in such an adenoviral vector, the E4 polyadenylation sequence alone or, most preferably, in combination with another sequence exists between the L5 fiber region and the right-side ITR, so as to sufficiently separate the retained L5 fiber region from the right-side ITR, such that viral production of such a vector approaches that of a singly replication deficient adenoviral vector, particularly a singly replication deficient E1 deficient adenoviral vector.

The spacer element can contain any sequence or sequences which are of a desired length, such as sequences at least about 15 base pairs (e.g., between about 15 base pairs and about 12,000 base pairs), preferably about 100 base pairs to about 10,000 base pairs, more preferably about 500 base pairs to about 8,000 base pairs, even more preferably about 1,500 base pairs to about 6,000 base pairs, and most preferably about 2,000 to about 3,000 base pairs in length. The spacer element sequence can be coding or non-coding and native or non-native with respect to the adenoviral genome, but does not restore the replication-essential function to the deficient region. The spacer can also contain a promoter-variable expression cassette. More preferably, the spacer comprises an additional polyadenylation sequence and/or a passenger gene. Preferably, in the case of a spacer inserted into a region deficient for E4, both the E4 polyadenylation sequence and the E4 promoter of the adenoviral genome or any other (cellular or viral) promoter remain in the vector. The spacer is located between the E4 polyadenylation site and the E4 promoter, or, if the E4 promoter is not present in the vector, the spacer is proximal to the right-side ITR. The spacer can comprise any suitable polyadenylation sequence. Examples of suitable polyadenylation sequences include synthetic optimized sequences, BGH (Bovine Growth Hormone), polyoma virus, TK (Thymidine Kinase), EBV (Epstein Barr Virus) and the papillomaviruses, including human papillomaviruses and BPV (Bovine Papilloma Virus). Preferably, particularly in the E4 deficient region, the spacer includes an SV40 polyadenylation sequence. The SV40 polyadenylation sequence allows for higher virus production levels of multiply replication deficient adenoviral vectors. In the absence of a spacer, production of fiber protein and/or viral growth of the multiply replication-deficient adenoviral vector is reduced by comparison to that of a singly replication-deficient adenoviral vector. However, inclusion of the spacer in at least one of the deficient adenoviral regions, preferably the E4 region, can counteract this decrease in fiber protein production and viral growth.

Although a passenger gene is typically inserted into the E1 deficient region of an adenoviral genome, a passenger gene can also function as the spacer in the E4 deficient region of the adenoviral genome. The passenger gene is limited only by the size of the fragment the vector can accommodate and can be any suitable gene. Examples of suitable passenger genes include marker gene sequences such as pGUS, secretory alkaline phosphatase, luciferase, B-galactosidase, and human anti-trypsin; therapeutic genes of interest such as the cystic fibrosis transmembrane regulator gene (CFTR); and potential immune modifiers such as B3-19K, E3-14.7, ICP47, fas ligand gene, and CTLA4 gene. Ideally, the spacer is composed of the glucuronidase gene. The use of a spacer in an adenoviral vector is described in, e.g., U.S. Pat. No. 5,851,806 and International Patent Application WO 97/21826.

Desirably, the adenoviral vector requires, at most, complementation of replication-essential gene functions of the E1, E2A, and/or E4 regions of the adenoviral genome for replication (i.e., propagation). However, the adenoviral genome can be modified to disrupt one or more replication-essential gene functions as desired by the practitioner, so long as the adenoviral vector remains deficient and can be propagated using, for example, complementing cells and/or exogenous DNA (e.g., helper adenovirus) encoding the disrupted replication-essential gene finctions. In this respect, the adenoviral vector can be deficient in replication-essential gene functions of only the early regions of the adenoviral genome, only the late regions of the adenoviral genome, and both the early and late regions of the adenoviral genome. The adenoviral vector also can have essentially the entire adenoviral genome removed, in which case it is preferred that at least the viral inverted terminal repeats (ITRs) and a packaging signal are left intact (i.e., an adenoviral amplicon). Suitable replication-deficient adenoviral vectors, including multiply replication-deficient adenoviral vectors, are disclosed in U.S. Patents 5,837,511, 5,851,806, 5,994,106, and 6,579,522, U.S. Published Patent Applications 2001/0043922 A1 2002/0004040 A1, 2002/0031831 A1, and 2002/0110545 A1, and International Patent Applications WO 95/34671, WO 97/12986, and WO 97/21826. Ideally, the pharmaceutical composition is virtually free of replication-competent adenovirus (RCA) contamination (e.g., the pharmaceutical composition comprises less than about 1% of RCA contamination). Most desirably, the pharmaceutical composition is RCA-free. Adenoviral vector compositions and stocks that are RCA-free are described in U.S. Pat. No. 5,944,106 and 6,482,616, U.S. Published Patent Application 2002/0110545 A1, and International Patent Application WO 95/34671. Ideally, the pharmaceutical composition also is free of E1-revertants when the adenoviral vector is E1-deficient in combination with deficiencies in other replication-essential gene finctions of another region of the adenoviral genome, as further described in International Patent Application WO 03/040314.

In addition to modification (e.g., deletion, mutation, or replacement) of adenoviral sequences encoding replication-essential gene functions, the adenoviral genome can contain benign or non-lethal modifications, i.e., modifications which do not render the adenovirus replication-deficient, or, desirably, do not adversely affect viral functioning and/or production of viral proteins, even if such modifications are in regions of the adenoviral genome that otherwise contain replication-essential gene functions. Such modifications commonly result from DNA manipulation or serve to facilitate expression vector construction. For example, it can be advantageous to remove or introduce restriction enzyme sites in the adenoviral genome. Such benign mutations often have no detectable adverse effect on viral functioning. For example, the adenoviral vector can comprise a deletion of nucleotides 10,594 and 10,595 (based on the adenoviral serotype 5 genome), which are associated with VA-RNA-1 transcription, but the deletion of which does not prohibit production of VA-RNA-1.

Similarly, the coat protein of a viral vector, preferably an adenoviral vector, can be manipulated to alter the binding specificity or recognition of a virus for a viral receptor on a potential host cell. For adenovirus, such manipulations can include deletion of regions of the fiber, penton, or hexon, insertions of various native or non-native ligands into portions of the coat protein, and the like. Manipulation of the coat protein can broaden the range of cells infected by a viral vector or enable targeting of a viral vector to a specific cell type. For example, in one embodiment, the expression vector is a viral vector comprising a chimeric coat protein (e.g., a fiber, hexon pIX, pIlIa, or penton protein), which differs from the wild-type (i.e., native) coat protein by the introduction of a nonnative amino acid sequence, preferably at or near the carboxyl terminus. Preferably, the nonnative amino acid sequence is inserted into or in place of an internal coat protein sequence. One of ordinary skill in the art will understand that the nonnative amino acid sequence can be inserted within the internal coat protein sequence or at the end of the internal coat protein sequence. The resultant chimeric viral coat protein is able to direct entry into cells of the viral, i.e., adenoviral, vector comprising the coat protein that is more efficient than entry into cells of a vector that is identical except for comprising a wild-type viral coat protein rather than the chimeric viral coat protein. Preferably, the chimeric virus coat protein binds a novel endogenous binding site present on the cell surface that is not recognized, or is poorly recognized by a vector comprising a wild-type coat protein. One direct result of this increased efficiency of entry is that the virus, preferably, the adenovirus, can bind to and enter numerous cell types which a virus comprising wild-type coat protein typically cannot enter or can enter with only a low efficiency.

In another embodiment of the invention, the expression vector is a viral vector comprising a chimeric virus coat protein not selective for a specific type of eukaryotic cell. The chimeric coat protein differs from the wild-type coat protein by an insertion of a nonnative amino acid sequence into or in place of an internal coat protein sequence. In this embodiment, the chimeric virus coat protein efficiently binds to a broader range of eukaryotic cells than a wild-type virus coat, such as described in International Patent Application WO 97/20051.

Specificity of binding of an adenovirus to a given cell can also be adjusted by use of an adenovirus comprising a short-shafted adenoviral fiber gene, as discussed in U.S. Pat. No. 5,962,311. Use of an adenovirus comprising a short-shafted adenoviral fiber gene reduces the level or efficiency of adenoviral fiber binding to its cell-surface receptor and increases adenoviral penton base binding to its cell-surface receptor, thereby increasing the specificity of binding of the adenovirus to a given cell. Alternatively, use of an adenovirus comprising a short-shafted fiber enables targeting of the adenovirus to a desired cell-surface receptor by the introduction of a nonnative amino acid sequence either into the penton base or the fiber knob.

Of course, the ability of a viral vector to recognize a potential host cell can be modulated without genetic manipulation of the coat protein. For instance, complexing an adenovirus with a bispecific molecule comprising a penton base-binding domain and a domain that selectively binds a particular cell surface binding site enables one of ordinary skill in the art to target the vector to a particular cell type.

Suitable modifications to a viral vector, specifically an adenoviral vector, are described in U.S. Pat. Nos. 5,543,328, 5,559,099, 5,712,136, 5,731,190, 5,756,086, 5,770,442, 5,846,782, 5,871,727, 5,885,808, 5,922,315, 5,962,311, 5,965,541, 6,057,155, 6,127,525, 6,153,435, 6,329,190, 6,455,314, and 6,465,253, U.S. Published Applications 2001/0047081 A1, 2002/0099024 A1, and 2002/0151027 A1, and International Patent Applications WO 95/02697, WO 95/16772, WO 95/34671, WO 96/07734, WO 96/22378, WO 96/26281, WO 97/20051, WO 98/07865, WO 98/07877, WO 98/40509, WO 98/54346, WO 00/15823, WO 01/58940, and WO 01/92549. Similarly, it will be appreciated that numerous expression vectors are available commercially. Construction of expression vectors is well understood in the art. Adenoviral vectors can be constructed and/or purified using methods known in the art (e.g., using complementing cell lines, such as the 293 cell line, Per.C6 cell line, or 293-ORF6 cell line) and methods set forth, for example, in U.S. Pat. Nos. 5,965,358, 5,994,128, 6,033,908, 6,168,941, 6,329,200, 6,383,795, 6,440,728, 6,447,995, and 6,475,757, U.S. Published Application 2002/0034735 A1, and International Patent Applications WO 98/53087, WO 98/56937, WO 99/15686, WO 99/54441, WO 00/12765, WO 01/77304, and WO 02/29388, as well as the other references identified herein. Adeno-associated viral vectors can be constructed and/or purified using the methods set forth, for example, in U.S. Pat. No. 4,797,368 and Laughlin et al., Gene, 23, 65-73 (1983).

The selection of expression vector for use in the present inventive method will depend on a variety of factors such as, for example, the host, immunogenicity of the vector, the desired duration of protein production, and the like. As each type of expression vector has distinct properties, a researcher has the freedom to tailor the present inventive method to any particular situation. Moreover, more than one type of expression vector can be used to deliver the nucleic acid sequence to the ocular cell. Thus, the invention provides a method of prophylactically or therapeutically treating an animal for at least one ocular-related disorder, wherein the method comprises contacting an ocular cell with different expression vectors, each comprising a nucleic acid sequence encoding an inhibitor of angiogenesis and/or a nucleic acid sequence encoding a neurotrophic agent. The nucleic acid sequence encoding the inhibitor of angiogenesis and/or the nucleic acid sequence encoding the neurotrophic agent are expressed, thereby resulting in the production of the inhibitor of angiogenesis and/or the neurotrophic agent to prophylactically or therapeutically treat the animal for an ocular-related disorder.

Preferably, at least two different types of expression vector (i.e., a plasmid and a viral vector or two different viral vectors) are delivered to the ocular cell. At least one expression vector can comprise a nucleic acid sequence encoding an inhibitor of angiogenesis. Similarly, at least one expression vector can comprise a nucleic acid sequence encoding a neurotrophic agent. Indeed, a mixture of expression vectors, some comprising the coding sequence for an inhibitor of angiogenesis and some comprising the coding sequence for a neurotrophic agent, can be administered. Desirably, at least one expression vector comprises the nucleic acid sequence encoding the inhibitor of angiogenesis and the nucleic acid sequence encoding the neurotrophic agent. More preferably, the nucleic acid sequence encoding the inhibitor of angiogenesis and the nucleic acid sequence encoding the neurotrophic agent are the same nucleic acid. Also preferably, the inhibitor of angiogenesis and the neurotrophic agent are a single factor. Preferably, the ocular cell is contacted with an adenoviral vector and an adeno-associated viral vector. One of ordinary skill in the art will appreciate the ability to capitalize on the advantageous properties of multiple delivery systems to treat or study ocular-related disorders.

One embodiment of the invention provides a method for prophylactically or therapeutically treating an animal for age-related macular degeneration. The method comprises contacting an ocular cell, such as an ocular cell associated with age-related macular degeneration, with an expression vector comprising a nucleic acid sequence encoding at least one inhibitor of angiogenesis and/or at least one neurotrophic factor. Alternatively, the ocular cell is contacted with at least two different types of expression vector, each expression vector comprising a nucleic acid sequence encoding at least one inhibitor of angiogenesis and/or at least one neurotrophic factor. As age-related macular degeneration commonly afflicts the elderly, preferably the expression vector is administered to an animal, i.e., a human, at least 55 years old. Age-related macular degeneration is a complex disease associated with a wide variety of complications affecting a number of ocular tissues. Ocular cells associated with age-related macular degeneration include, but are not limited to, cells of neural origin, cells of all layers of the retina, especially retinal pigment epithelial cells, glial cells, and pericytes. Other ocular cells that are suitable for use in the method of the invention include, for example, endothelial cells, iris epithelial cells, corneal cells, ciliary epithelial cells, Mueller cells, astrocytes, muscle cells surrounding and attached to the eye (e.g., cells of the lateral rectus muscle), fibroblasts (e.g., fibroblasts associated with the episclera), orbital fat cells, cells of the sclera and episclera, connective tissue cells, muscle cells, and cells of the trabecular meshwork. The trabecular meshwork is associated with the passage for fluid drainage out of the eye. Other cells linked to various ocular-related diseases include, for example, fibroblasts and vascular endothelial cells. In that a great deal of retinal damage occurs as a result of edema, thickening of underlying membranes, and build-up of metabolic byproducts, preferably the expression vector is administered to an area of vascular leakage.

The nucleic acid sequence is desirably present as part of an expression cassette, i.e., a particular nucleotide sequence that possesses functions which facilitate subcloning and recovery of a nucleic acid sequence (e.g., one or more restriction sites) or expression of a nucleic acid sequence (e.g., polyadenylation or splice sites). The nucleic acid sequence is preferably located in the E1 region (e.g., replaces the E1 region in whole or in part) of the adenoviral genome. For example, the E1 region can be replaced by a promoter-variable expression cassette comprising the nucleic acid sequence(s). The expression cassette is preferably inserted in a 3′-5′ orientation, e.g., oriented such that the direction of transcription of the expression cassette is opposite that of the surrounding adjacent adenoviral genome. In addition to the expression cassette comprising the nucleic acid sequence(s), the adenoviral vector can comprise other expression cassettes containing nucleic acid sequences encoding other products, which cassettes can replace any of the deleted regions of the adenoviral genome. The insertion of an expression cassette into the adenoviral genome (e.g., into the E1 region of the genome) can be facilitated by known methods, for example, by the introduction of a unique restriction site at a given position of the adenoviral genome. As set forth above, preferably all or part of the E3 region of the adenoviral vector also is deleted.

According to the invention, the nucleic acid sequence is operably linked to regulatory sequences necessary for expression, i.e., a promoter. A “promoter” is a DNA sequence that directs the binding of RNA polymerase and thereby promotes RNA synthesis. A nucleic acid sequence is “operably linked” to a promoter when the promoter is capable of directing transcription of that nucleic acid sequence. A promoter can be native or non-native to the nucleic acid sequence to which it is operably linked.

Any promoter (i.e., whether isolated from nature or produced by recombinant DNA or synthetic techniques) can be used in connection with the invention to provide for transcription of the nucleic acid sequence. The promoter preferably is capable of directing transcription in a eukaryotic (desirably mammalian) cell. The functioning of the promoter can be altered by the presence of one or more enhancers and/or silencers present on the vector. “Enhancers” are cis-acting elements of DNA that stimulate or inhibit transcription of adjacent genes. An enhancer that inhibits transcription also is termed a “silencer.” Enhancers differ from DNA-binding sites for sequence-specific DNA binding proteins found only in the promoter (which also are termed “promoter elements”) in that enhancers can function in either orientation, and over distances of up to several kilobase pairs (kb), even from a position downstream of a transcribed region.

A comparison of promoter sequences that function in eukaryotes has revealed conserved sequence elements. Generally, eukaryotic promoters transcribed by RNA polymerase II are typified by a “TATA box” centered at approximately position −25, which appears to be essential for accurately positioning the start of transcription. The TATA box directs RNA polymerase to begin transcribing approximately 30 base pairs (bp) downstream in mammalian systems. The TATA box finctions in conjunction with at least two other upstream sequences located about 40 bp and 110 bp upstream of the start of transcription. Typically, a so-called “CCAAT box” serves as one of the two upstream sequences, and the other often is a GC-rich segment. The CCAAT homology can reside on different strands of the DNA. The upstream promoter element also can be a specialized signal such as one of those which have been described in the art and which appear to characterize a certain subset of genes.

To initiate transcription, the TATA box and the upstream sequences are each recognized by regulatory proteins which bind to these sites, and activate transcription by enabling RNA polymerase II to bind the DNA segment and properly initiate transcription. Whereas base changes outside the TATA box and the upstream sequences have little effect on levels of transcription, base changes in either of these elements substantially lower transcription rates (see, e.g., Myers et al., Science, 229, 242-247 (1985); McKnight et al., Science, 217, 316-324 (1982)). The position and orientation of these elements relative to one another, and to the start site, are important for the efficient transcription of some, but not all, coding sequences. For instance, some promoters function well in the absence of any TATA box. Similarly, the necessity of these and other sequences for promoters recognized by RNA polymerase I or III, or other RNA polymerases, can differ.

Accordingly, promoter regions can vary in length and sequence and can further encompass one or more DNA binding sites for sequence-specific DNA binding proteins and/or an enhancer or silencer. Enhancers and/or silencers can similarly be present on a nucleic acid sequence outside of the promoter per se. Desirably, a cellular or viral enhancer, such as the cytomegalovirus (CMV) immediate-early enhancer, is positioned in the proximity of the promoter to enhance promoter activity. In addition, splice acceptor and donor sites can be present on a nucleic acid sequence to enhance transcription.

The invention preferentially employs a viral promoter. Suitable viral promoters are known in the art and include, for instance, cytomegalovirus (CMV) promoters, such as the CMV immediate-early promoter, promoters derived from human immunodeficiency virus (HIV), such as the HIV long terminal repeat promoter, Rous sarcoma virus (RSV) promoters, such as the RSV long terminal repeat, mouse mammary tumor virus (MMTV) promoters, HSV promoters, such as the Lap2 promoter or the herpes thymidine kinase promoter (Wagner et al., Proc. Natl. Acad. Sci., 78, 144-145 (1981)), promoters derived from SV40 or Epstein Barr virus, an adeno-associated viral promoter, such as the p5 promoter, and the like. Preferably, the viral promoter is an adenoviral promoter, such as the Ad2 or Ad5 major late promoter and tripartite leader, a CMV promoter, or an RSV promoter.

Alternatively, the invention employs a cellular promoter, i.e., a promoter that drives expression of a cellular protein. Preferred cellular promoters for use in the invention will depend on the desired expression profile to produce the therapeutic agent(s). In one aspect, the cellular promoter is preferably a constitutive promoter that works in a variety of cell types, such as cells associated with the eye. Suitable constitutive promoters can drive expression of genes encoding transcription factors, housekeeping genes, or structural genes common to eukaryotic cells. For example, the Ying Yang 1 (YY1) transcription factor (also referred to as NMP-1, NF-E1, and UCRBP) is a ubiquitous nuclear transcription factor that is an intrinsic component of the nuclear matrix (Guo et al., PNAS, 92, 10526-10530 (1995)). YY1 is a regulatory protein that responds to changes in the cellular environment. Accordingly, the viral infection process can upregulate the activity of the YY1 promoter to provide for enhanced transcription and, subsequently, enhanced protein production from the viral construct. While the promoters described herein are considered as constitutive promoters, it is understood in the art that constitutive promoters can be upregulated. Promoter analysis shows that the elements critical for basal transcription reside from −277 to +475 of the YY1 gene relative to the transcription start site from the promoter, and include a TATA and CCAAT box. JEM-1 (also known as HGMW and BLZF-1) also is a ubiquitous nuclear transcription factor identified in normal and tumorous tissues (Tong et al., Leukemia, 12(11), 1733-1740 (1998), and Tong et al., Genomics, 69(3), 380-390 (2000)). JEM-1 is involved in cellular growth control and maturation, and can be upregulated by retinoic acids. Sequences responsible for maximal activity of the JEM-1 promoter has been located at −432 to +101 of the JEM-1 gene relative the transcription start site of the promoter. Unlike the YY1 promoter, the JEM-1 promoter does not comprise a TATA box. The ubiquitin promoter, specifically UbC, is a strong constitutively active promoter functional in several species. The UbC promoter is further characterized in Marinovic et al., J Biol. Chem., 277(19), 16673-16681 (2002).

Many of the above-described promoters are constitutive promoters. Instead of being a constitutive promoter, the promoter can be an inducible promoter, i.e., a promoter that is up- and/or down-regulated in response to appropriate signals. For instance, the regulatory sequences can comprise a hypoxia driven promoter, which is active when the ocular neovascularization or age-related macular degeneration is associated with hypoxia. Other examples of suitable inducible promoter systems include, but are not limited to, the IL-8 promoter, the metallothionine inducible promoter system, the bacterial lacZYA expression system, the tetracycline expression system, and the T7 polymerase system. Further, promoters that are selectively activated at different developmental stages (e.g., globin genes are differentially transcribed from globin-associated promoters in embryos and adults) can be employed. The promoter sequence that regulates expression of the nucleic acid sequence can contain at least one heterologous regulatory sequence responsive to regulation by an exogenous agent. The regulatory sequences are preferably responsive to exogenous agents such as, but not limited to, drugs, hormones, or other gene products (ideally gene products produced in the eye). For example, the regulatory sequences, e.g., promoter, preferably are responsive to glucocorticoid receptor-hormone complexes, which, in turn, enhance the level of transcription of a therapeutic gene or a therapeutic fragment thereof.

Preferably, the regulatory sequences comprise a tissue-specific promoter, i.e., a promoter that is preferentially activated in a given tissue and results in expression of a gene product in the tissue where activated. A typically used tissue-specific promoter is a myocyte-specific promoter. A promoter exemplary of a myocyte-specific promoter is the myosin light-chain 1A promoter. A tissue-specific promoter for use in the present inventive vector can be chosen by the ordinarily skilled artisan based upon the target tissue or cell-type. Preferred tissue-specific promoters for use in the present inventive methods are specific to ocular tissue, such as a rhodopsin promoter. Examples of rhodopsin promoters include, but are not limited to, a GNAT cone-transducing alpha-subunit gene promoter or an interphotoreceptor retinoid binding protein promoter.

One of ordinary skill in the art will appreciate that each promoter drives transcription, and, therefore, protein expression, differently with respect to time and amount of protein produced. For example, the CMV promoter is characterized as having peak activity shortly after transduction, i.e., about 24 hours after transduction, then quickly tapering off. The cellular promoters described herein display different expression profiles which can be exploited to optimize production of the therapeutic factor(s). The UbC and YY1 promoters drive steady expression of transgenes for a prolonged period of time compared to the CMV promoter, which is associated with a rapid loss of transcription compared to transcription levels observed at one day post-administration of the vector. In one aspect, the promoter of the invention preferably drives transcription of the nucleic acid sequence encoding the therapeutic factor(s) or fragment(s) thereof without a substantial loss of activity at about one month (28 days) post-administration (preferably 35 days, 42 days or 48 days post-administration) when administered intraocularly (e.g., intravitreously) to a mouse at a dose of about 2×10⁸ particles. Preferably, the level of transcription of the nucleic acid sequence (which ideally results in protein production) is not diminished more than 10-fold (e.g., no more than 7-fold) at 28 days compared to the level of transcription of the nucleic acid sequence at one day post-administration. More preferably, the level of transcription is not diminished more than 5-fold (e.g., no more than 3-fold) at 28 days compared to the level of transcription at one day post-administration of the expression (e.g., adenoviral) vector. Most preferably, there is no loss of promoter activity at 28 days. Ideally, the same levels of transcription are achieved in the eye of a human. Some cellular promoters show increased expression over time compared to levels at one day post-administration. The day 1 levels may have a low initial level of activity (i.e., initial expression levels are minimally above background). For example, initial expression from the JEM-1 promoter is near background levels. However, the level of transcription is increased by 10-fold at 14 days post-transduction compared to the initial level of transcription and remains elevated at 28 days post-vector administration. Thus, a promoter can be selected for use in the methods of the invention by matching its particular pattern of activity with the desired pattern and level of expression of at least one inhibitor of angiogenesis and/or at least one neurotrophic factor.

In summary, the invention provides a method of prophylactically or therapeutically treating an animal for an ocular-related disorder. The method comprises contacting an ocular cell with an adenoviral vector comprising a nucleic acid sequence operably linked to a cellular promoter and encoding an inhibitor of angiogenesis and/or a neurotrophic agent, thereby resulting in the production of the inhibitor of angiogenesis and/or the neurotrophic agent to prophylactically or therapeutically treat the animal for an ocular-related disorder, with the proviso that if the adenoviral vector is administered to a mouse at a dose of 2×10⁸ particles, the level of transcription of the nucleic acid sequence is not diminished more than ten-fold at 28 days post-administration of the adenoviral vector compared to the level of transcription of the nucleic acid sequence at one day post-administration of the adenoviral vector.

Alternatively, a hybrid promoter can be constructed which combines the desirable aspects of multiple promoters. For example, a CMV-UbC hybrid promoter combining the CMV promoter's high activity with the UbC promoter's high maintenance level of activity would be especially preferred for use in many embodiments of the present inventive method. It is also possible to select a promoter with an expression profile that can be manipulated by an investigator.

Also preferably, the expression vector comprises a nucleic acid encoding a cis-acting factor, wherein the cis-acting factor modulates the expression of the nucleic acid sequence. Preferably, the cis-acting factor comprises matrix attachment region (MAR) sequences (e.g., immunoglobulin heavy chain (Jenunwin et al., Nature, 385(16), 269 (1997)), apolipoprotein B, or locus control region (LCR) sequences, among others. MAR sequences have been characterized as DNA sequences that associate with the nuclear matrix after a combination of nuclease digestion and extraction (Bode et al., Science, 255(5041), 195-197 (1992)). MAR sequences are often associated with enhancer-type regulatory regions and, when integrated into genomic DNA, MAR sequences augment transcriptional activity of adjacent nucleotide sequences. It has been postulated that MAR sequences play a role in controlling the topological state of chromatin structures, thereby facilitating the formation of transcriptionally-active complexes. Similarly, it is believed LCR sequences function to establish and/or maintain domains permissive for transcription. Many LCR sequences give tissue specific expression of associated nucleic acid sequences. Addition of MAR or LCR sequences to the expression vector can further enhance expression of at least one inhibitor of angiogenesis and/or at least one neurotrophic factor.

With respect to promoters, nucleic acid sequences, selectable markers, and the like, located on an expression vector according to the invention, such elements can be present as part of a cassette, either independently or coupled. As further described herein, a “cassette” is a particular base sequence that possesses functions which facilitate subcloning and recovery of nucleic acid sequences (e.g., one or more restriction sites) or expression (e.g., polyadenylation or splice sites) of particular nucleic acid sequences.

Construction of an exogenous nucleic acid operably linked to regulatory sequences necessary for expression is well within the skill of the art (see, for example, Sambrook et al., Molecular Cloning: A Laboratory Manual, 2d ed. (1989)). With respect to the expression of nucleic acid sequences according to the invention, the ordinary skilled artisan is aware that different genetic signals and processing events control levels of nucleic acids and proteins/peptides in a cell, such as, for instance, transcription, mRNA translation, and post-transcriptional processing. Transcription of DNA into RNA requires a functional promoter, as described herein.

Protein expression is dependent on the level of RNA transcription that is regulated by DNA signals, RNA stability, and the levels of DNA template. Similarly, often translation of mRNA requires, at the very least, an AUG initiation codon, which is usually located within 10 to 100 nucleotides of the 5′ end of the message. Sequences flanking the AUG initiator codon have been shown to influence its recognition by eukaryotic ribosomes, with conformity to a perfect Kozak consensus sequence resulting in optimal translation (see, e.g., Kozak, J Molec. Biol., 196, 947-950 (1987)). Also, successful expression of an exogenous nucleic acid in a cell can require post-translational modification of a resultant protein. Thus, production of a protein can be affected by the efficiency with which DNA (or RNA) is transcribed into mRNA, the efficiency with which mRNA is translated into protein, and the ability of the cell to carry out post-translational modification. These are all factors of which the ordinary skilled artisan is aware and is capable of manipulating using standard means to achieve the desired end result.

Along these lines, to optimize protein production, preferably the nucleic acid sequence further comprises a polyadenylation site following the coding region of the nucleic acid sequence. Also, preferably all the proper transcription signals (and translation signals, where appropriate) will be correctly arranged such that the nucleic acid sequence will be properly expressed in the cells into which it is introduced. If desired, the nucleic acid sequence also can incorporate splice sites (i.e., splice acceptor and splice donor sites) to facilitate mRNA production. Moreover, if the nucleic acid sequence encodes a protein or peptide, which is a processed or secreted protein or acts intracellularly, preferably the nucleic acid sequence further comprises the appropriate sequences for processing, secretion, intracellular localization, and the like.

In certain embodiments, it may be advantageous to modulate expression of the at least one inhibitor of angiogenesis and/or at least one neurotrophic factor. An especially preferred method of modulating expression of a nucleic acid sequence comprises addition of site-specific recombination sites on the expression vector. Contacting an expression vector comprising site-specific recombination sites with a recombinase will either up- or down-regulate transcription of a coding sequence, or simultaneously up-regulate transcription one coding sequence and down-regulate transcription of another, through the recombination event. Use of site-specific recombination to modulate transcription of a nucleic acid sequence is described in, for example, U.S. Pat. Nos. 5,801,030 and 6,063,627 and International Patent Application WO 97/09439.

Preferably, the expression vector of the present inventive method comprises a nucleic acid encoding an inhibitor of angiogenesis. More preferably, the nucleic acid sequence encodes multiple inhibitors of angiogenesis. By “inhibitor of angiogenesis” is meant any factor that prevents or ameliorates neovascularization. One of ordinary skill in the art will understand that complete prevention or amelioration of neovascularization is not required in order to realize a therapeutic effect. Therefore, the present inventive methods contemplate both partial and complete prevention and amelioration of angiogenesis. An inhibitor of angiogenesis includes, for instance, an anti-angiogenic factor, an anti-sense molecule specific for an angiogenic factor, a ribozyme, a small interfering RNA (siRNA, an RNA interfering molecule), a receptor for an angiogenic factor, and an antibody that binds a receptor for an angiogenic factor.

The anti-angiogenic factors contemplated for use in the invention include pigment epithelium-derived factor, angiostatin, vasculostatin, endostatin, platelet factor 4, heparinase, interferons (e.g., INFα), tissue inhibitor of metalloproteinase 3 (TIMP3), and the like. Such factors prevent the growth of new blood vessels, promote vessel maturation, inhibit permeability of blood vessels, inhibit the migration of endothelial cells, and the like. Various anti-angiogenic factors are described in International Patent Application No. WO 02/22176. One of ordinary skill in the art will appreciate that any anti-angiogenic factor can be modified or truncated and retain anti-angiogenic activity. As such, active fragments of anti-angiogenic factors (i.e., those fragments having biological activity sufficient to inhibit angiogenesis) are also suitable for use in the present inventive methods.

An anti-sense molecule specific for an angiogenic factor should generally be substantially identical to at least a portion, preferably at least about 20 continuous nucleotides, of the nucleic acid encoding the angiogenic factor to be inhibited, but need not be identical. The anti-sense nucleic acid molecule can be designed such that the inhibitory effect applies to other proteins within a family of genes exhibiting homology or substantial homology to the nucleic acid. The introduced anti-sense nucleic acid molecule also need not be full-length relative to either the primary transcription product or fully processed mRNA. Generally, higher homology can be used to compensate for the use of a shorter sequence. Furthermore, the anti-sense molecule need not have the same intron or exon pattern, and homology of non-coding segments will be equally effective. Antisense phosphorothiotac oligodeoxynucleotides (PS-ODNs) is exemplary of an anti-sense molecule specific for an angiogenic factor. Also suitable are other RNA interfering agents, such as siRNA (see, e.g., Chui et al., Mol. Cell., 10(3), 549-61 (2002)).

Ribozymes can be designed that specifically pair with virtually any target RNA and cleave the phosphodiester backbone at a specific location, thereby functionally inactivating the target RNA. In carrying out this cleavage, the ribozyme is not itself altered and is, thus, capable of recycling and cleaving other molecules, making it a true enzyme. The inclusion of ribozyme sequences within antisense RNAs confers RNA-cleaving activity upon them, thereby increasing the activity of the constructs. The design and use of target RNA-specific ribozymes is described in Haseloff et al., Nature, 334, 585-591 (1988). Preferably, the ribozyme comprises at least about 20 continuous nucleotides complementary to the target sequence on each side of the active site of the ribozyme.

Receptors specific for angiogenic factors inhibit neovascularization by sequestering growth factors away from functional receptors capable of promoting a cellular response. For example, Flt and Flk receptors, as well as VEGF-receptor chimeric proteins, compete with VEGF receptors on vascular endothelial cells to inhibit endothelial cell growth (Aiello, PNAS, 92, 10457 (1995)). Also contemplated are growth factor-specific antibodies and fragments thereof (e.g., Fab, F(ab′)₂, and Fv) that neutralize angiogenic factors or bind receptors for angiogenic factors.

The invention also contemplates delivery of a nucleic acid sequence encoding at least one neurotrophic agent (or neurotrophic factor) to ocular cells or cells associated with age-related macular degeneration. Neurotrophic factors are thought to be responsible for the maturation of developing neurons and for maintaining adult neurons. Thus, the methods and materials of the invention can be used to inhibit or reverse neural cell degeneration and death not associated with neovascular diseases. Neurotrophic factors are divided into three subclasses: neuropoietic cytokines; neurotrophins; and the fibroblast growth factors. Ciliary neurotrophic factor (CNTF) is exemplary of neuropoietic cytokines. CNTF promotes the survival of ciliary ganglionic neurons and supports certain neurons that are NGF-responsive. Neurotrophins include, for example, brain-derived neurotrophic factor and nerve growth factor, perhaps the best characterized neurotrophic factor. Other neurotrophic factors suitable for being encoded by the nucleic acid sequence of the present inventive methods include, for example, transforming growth factors, glial cell-line derived neurotrophic factor, neurotrophin 3, neurotrophin 4/5, and interleukin 1-β. Neurotrophic factors associated with angiogenesis, such as aFGF and bFGF, are less preferred. The neurotrophic factor of the present inventive method can also be a neuronotrophic factor, e.g., a factor that enhances neuronal survival. It has been postulated that neurotrophic factors can actually reverse degradation of neurons. Such factors, conceivably, are useful in treating the degeneration of neurons associated with vision loss. Neurotrophic factors function in both paracrine and autocrine fashions, making them ideal therapeutic agents. Preferably, the nucleic acid sequence of the invention encodes both an inhibitor of angiogenesis and a neurotrophic factor. More preferably, the nucleic acid sequence encodes at least one factor comprising both anti-angiogenic and neurotrophic properties. Most preferably, the factor comprising both anti-angiogenic and neurotrophic properties is pigment epithelium-derived factor (PEDF).

PEDF, also named early population doubling factor-1 (EPC-1), is a secreted protein having homology to a family of serine protease inhibitors named serpins. PEDF is made predominantly by retinal pigment epithelial cells and is detectable in most tissues and cell types of the body. PEDF has been observed to induce differentiation in retinoblastoma cells and enhance survival of neuronal populations (Chader, Cell Different., 20, 209-216 (1987)). Factors that enhance neuronal survival under adverse conditions, such as PEDF, are termed “neuronotrophic,” as described herein. PEDF further has gliastatic activity, or has the ability to inhibit glial cell growth. As discussed above, PEDF also has anti-angiogenic activity. Anti-angiogenic derivatives of PEDF include SLED proteins, discussed in WO 99/04806. It has also been postulated that PEDF is involved with cell senescence (Pignolo et al., J Biol. Chem., 268(12), 8949-8957 (1998)). PEDF for use in the present inventive method can be derived from any source, and is further characterized in U.S. Pat. No. 5,840,686 and International Patent Applications WO 93/24529 and WO 99/04806.

In addition to the methods of prophylactically or therapeutically treating an ocular-related disorder, the invention further provides a viral vector comprising a nucleic acid sequence encoding PEDF or a therapeutic fragment thereof, wherein the nucleic acid sequence operably linked to regulatory sequences necessary for expression of PEDF or a therapeutic fragment thereof. The nucleic acid sequence can be obtained from any source, e.g., isolated from nature, synthetically generated, isolated from a genetically engineered organism, and the like. Appropriate viral vectors and regulatory sequences are discussed herein. In nature, PEDF is generated in human fetus retinal cells. The viral vector of the invention can be used to create sufficient amounts of recombinant PEDF or can be used in methods of research or treatment, e.g., the present inventive method. Preferably, the viral vector is an adenoviral vector, as described herein, comprising a nucleic acid sequence encoding PEDF. More preferably, the adenoviral vector comprises the nucleic acid sequence set forth in SEQ ID NO: 1.

The expression vector, e.g., the adenoviral or the adeno-associated viral vector, also can comprise a nucleic acid sequence encoding a therapeutic fragment of at least one inhibitor of angiogenesis or at least one neurotrophic factor. One of ordinary skill in the art will appreciate that any inhibitor of angiogenesis or neurotrophic factor, e.g., PEDF, can be modified or truncated and retain anti-angiogenic or neurotrophic activity. As such, therapeutic fragments (i.e., those fragments having biological activity sufficient to, for example, inhibit angiogenesis or promote neuron survival) also are suitable for incorporation into the expression vector. Also suitable for incorporation into the expression vector are nucleic acid sequences comprising substitutions, deletions, or additions, but which encode a functioning inhibitor of angiogenesis or neurotrophic factor or a therapeutic fragment of any of the foregoing. Likewise, a fusion protein comprising an anti-angiogenic factor or neurotrophic factor or a therapeutic fragment thereof and for example, a moiety that stabilizes peptide conformation, also can be present in the expression vector. A functioning inhibitor of angiogenesis or a therapeutic fragment thereof prevents or ameliorates neovascularization. A functioning neurotrophic factor or a therapeutic fragment thereof desirably promotes neuronal cell differentiation, inhibits glial cell proliferation, and/or promotes neuronal cell survival. One of ordinary skill in the art will understand that complete prevention or amelioration of neovascularization is not required in order to realize a therapeutic effect. Likewise, complete induction of neuron survival or differentiation is not required in order to realize a benefit. Therefore, both partial and complete prevention and amelioration of angiogenesis or promotion of neuron survival is appropriate. The ordinarily skilled artisan has the ability to determine whether a modified therapeutic factor or a fragment thereof has neurotrophic and anti-angiogenic therapeutic activity using, for example, neuronal cell differentiation and survival assays (see, for example, U.S. Pat. No. 5,840,686), the mouse ear model of neovascularization, or the rat hindlimb ischemia model.

Similarly, one of ordinary skill in the art will appreciate that the inhibitor of angiogenesis and/or the neurotrophic factor can be a factor that acts upon a receptor for an anti-angiogenic factor or a receptor for a neurotrophic factor, thereby resulting in the desired biological effect. For instance, the expression vector can comprise a nucleic acid sequence encoding an antibody or peptide agonist that binds and activates the PEDF receptor, which signals a series of intracellular events responsible for the biological activity of PEDF. Likewise, the expression vector can comprise a nucleic acid sequence encoding a peptide that interacts with a PEDF receptor to achieve a biological effect. For example, a dominant positive protein can be constructed which constitutively activates cell-signaling via the PEDF receptor. For a discussion of PEDF receptors, see, for example, Alberdi et al., J Biol. Chem., 274(44), 31605 (1999).

The invention also contemplates the use of nucleic acid sequences encoding chimeric or fusion peptides in the present inventive method. Through recombinant DNA technology, scientists have been able to generate fusion proteins that contain the combined amino acid sequence of two or more proteins. The ordinarily skilled artisan can fuse the active domains of two or more factors to generate chimeric peptides with desired activity. The chimeric peptide can comprise the entire amino acid sequences of two or more peptides or, alternatively, can be constructed to comprise portions of two or more peptides (e.g., 10, 20, 50, 75, 100, 400, 500, or more amino acid residues). Desirably, the chimeric peptide comprises anti-angiogenic and neurotrophic activity, which can be determined using routine methods.

As discussed herein, the expression vector of the present inventive method comprises a nucleic acid sequence that encodes at least one inhibitor of angiogenesis and/or at least one neurotrophic factor. Therefore, the nucleic acid sequence can encode multiple, i.e., two, three, or more, inhibitors of angiogenesis. Likewise, the nucleic acid sequence can encode multiple, i.e., two, three, or more, neurotrophic factors. In a preferred embodiment, the nucleic acid sequence encodes PEDF and ciliary neurotrophic factor (CNTF). Also preferably, the nucleic acid sequence encodes at least one inhibitor of angiogenesis and at least one neurotrophic factor. Multiple inhibitors of angiogenesis and/or multiple neurotrophic factors can be operably linked to different promoters. As discussed herein, different promoters have dissimilar levels and patterns of activity. One of ordinary skill in the art will appreciate the freedom to dictate the expression of different coding sequences through the use of multiple promoters. Alternatively, the multiple coding sequences can be operably linked to the same promoter to form a polycistronic element. The polycistronic element is transcribed into a single mRNA molecule when transduced into the ocular cell. Translation of the mRNA molecule is initiated at each coding sequence, thereby producing the multiple, separate peptides simultaneously. The invention also contemplates contacting an ocular cell or a cell associated with age-related macular degeneration with a cocktail of expression vectors, wherein each expression vector encodes a different inhibitor of angiogenesis and/or neurotrophic factor. The cocktail of expression vectors can further comprise different types of expression vectors, e.g., adenoviral vectors and adeno-associated viral vectors.

The methods of the invention can be part of a treatment regimen involving other therapeutic modalities. It is appropriate, therefore, if the ocular-related disorder, namely ocular neovascularization or age-related macular degeneration, has been treated, is being treated, or will be treated with any of a number of ocular therapies, such as drug therapy, photodynamic therapy, photocoagulation laser therapy, panretinal therapy, thermotherapy, radiation therapy, or surgery. Preferably, the surgery is macular translocation, removal of subretinal blood, or removal of subretinal choroidal neovascular membrane. The expression vector is preferably administered intraocularly for the prophylactic or therapeutic treatment of age-related macular degeneration or persistent or recurrent ocular neovascularization treated with drugs, surgery, laser photocoagulation, and photodynamic therapies.

The expression vector is preferably administered as soon as possible after it has been determined that an animal, such as a mammal, specifically a human, is at risk for ocular neovascularization or age-related macular degeneration (prophylactic treatment) or has begun to develop ocular neovascularization or age-related macular degeneration (therapeutic treatment). Treatment will depend, in part, upon the particular nucleic acid sequence used, the particular inhibitor of angiogenesis and/or neurotrophic factor expressed from the nucleic acid sequence, the route of administration, and the cause and extent, if any, of ocular neovascularization or age-related macular degeneration realized. For example, systemic administration or administration to both eyes is preferred in the prophylactic treatment of macular degeneration because, once one eye is affected, the other eye is at risk (up to 19% per year).

The expression vector of the present invention desirably is administered in a pharmaceutical composition, which comprises a pharmaceutically acceptable carrier and the expression vector(s). Any suitable pharmaceutically acceptable carrier can be used within the context of the invention, and such carriers are well known in the art. The choice of carrier will be determined, in part, by the particular site to which the composition is to be administered and the particular method used to administer the composition.

Suitable formulations include aqueous and non-aqueous solutions, isotonic sterile solutions, which can contain anti-oxidants, buffers, bacteriostats, and solutes that render the formulation isotonic with the blood or intraocular fluid of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid carrier, for example, water, immediately prior to use. Extemporaneous solutions and suspensions can be prepared from sterile powders, granules, and tablets of the kind previously described. Preferably, the pharmaceutically acceptable carrier is a buffered saline solution. More preferably, the expression vector for use in the inventive method is administered in a pharmaceutical composition formulated to protect and/or stabilize the expression vector from damage prior to administration. For example, the pharmaceutical composition can be formulated to reduce loss of the expression vector on devices used to prepare, store, or administer the expression vector, such as glassware, syringes, pellets, slow-release devices, pumps, or needles. The pharmaceutical composition can be formulated to decrease the light sensitivity and/or temperature sensitivity of the expression vector. To this end, the pharmaceutical composition preferably comprises a pharmaceutically acceptable liquid carrier, such as, for example, those described above, and a stabilizing agent selected from the group consisting of polysorbate 80, L-arginine, polyvinylpyrrolidone, trehalose, and combinations thereof. In one embodiment, the formulation comprises Tris base (10 mM), NaCl (75 mM), MgCl.6H₂O (1 mM), polysorbate 80 (0.0025%) and trehalose dehydrate (5%). Use of such a pharmaceutical composition will extend the shelf life of the vector, facilitate administration, and increase the efficiency of the present inventive methods. In this regard, a pharmaceutical composition also can be formulated to enhance transduction efficiency. Suitable compositions are further described in U.S. Pat. Nos. 6,225,289 and 6,514,943.

In addition, one of ordinary skill in the art will appreciate that the expression vector, e.g., viral vector, of the invention can be present in a composition with other therapeutic or biologically-active agents. For example, therapeutic factors useful in the treatment of a particular indication can be present. For instance, if treating vision loss, hyaluronidase can be added to a composition to, for example, effect the break down of blood and blood proteins in the vitreous of the eye. Factors that control inflammation, such as ibuprofen or steroids, can be part of the composition to reduce swelling and inflammation associated with in vivo administration of the viral vector and ocular distress. Inflammation also can be controlled by down-regulating the effects of cytokines involved in the inflammation process (e.g., TNFα). Alternatively, agonists for chemokines which control inflammation (e.g., TGFβ) can be included to reduce the harmful effects of inflammation. Immune system suppressors can be administered in combination with the present inventive method to reduce any immune response to the vector itself or associated with an ocular disorder. Anti-angiogenic factors, such as soluble growth factor receptors (sflt), growth factor antagonists (e.g., angiotensin), an anti-growth factor antibody (e.g., Lucentis™), Squalamine (an aminosterol), and the like also can be part of the composition, as well as additional neurotrophic factors. Similarly, vitamins and minerals, anti-oxidants, and micronutrients can be co-administered. Antibiotics, i.e., microbicides and fungicides, can be present to reduce the risk of infection associated with gene transfer procedures and other disorders. Ligands for nuclear receptors such as thyroid hormones, retinoids, specific prostaglandins, estrogen hormone, glucocorticoids or their analogues can be part of the composition. Small molecule agonists for the PEDF receptor also can be included in the formulation. Such small molecule agonists can amplify the therapeutic effect of the inventive method. Suitable drugs for inclusion in the formulation include, but are not limited to, a prostaglandin analogue, a beta-blocker (as commonly used for glaucoma treatment), hyaluronidase (e.g., Vitrase™ available from Allergan), pegaptanib sodium (e.g., Macugen™), tetrahydrozoline hydrochloride (e.g., Visine™), dorzolamide hydrochloride (Cosopt™ and Truspot™), and an alpha-2-adrenergic agonist (e.g., Alphagan™). Alternatively, these compounds can be administered separately to the animal.

One skilled in the art will appreciate that suitable methods, i.e., invasive and noninvasive methods, of administering an expression vector whereon the expression vector will contact an ocular cell are available. Although more than one route can be used to administer a particular expression vector, a particular route can provide a more immediate and more effective reaction than another route. Accordingly, the described routes of administration are merely exemplary and are in no way limiting.

The present inventive methods are not dependent on the mode of administering the expression vector to an animal, preferably a human, to achieve the desired effect. As such, any route of administration is appropriate so long as the expression vector contacts an appropriate ocular cell. The expression vector for use in the present inventive methods can be appropriately formulated and administered in the form of an injection, eye lotion, ointment, implant and the like. The expression vector can be applied, for example, systemically, topically, intracamerally, subconjunctivally, intraocularly, retrobulbarly, periocularly (e.g., subtenon delivery), subretinally, or suprachoroidally. In certain cases, it may be appropriate to administer multiple applications and employ multiple routes, e.g., subretinal and intravitreous, to ensure sufficient exposure of ocular cells to the expression vector. Multiple applications of the expression vector may also be required to achieve the desired effect.

Depending on the particular case, it may be desirable to non-invasively administer the expression vector to a patient. For instance, if multiple surgeries have been performed, the patient displays low tolerance to anesthetic, or if other ocular-related disorders exist, topical administration of the expression vector may be most appropriate. Topical formulations are well known to those of skill in the art. Such formulations are suitable in the context of the invention for application to the skin. The use of patches, corneal shields (see, e.g., U.S. Pat. No. 5,185,152), and ophthalmic solutions (see, e.g., U.S. Pat. No. 5,710,182) and ointments, e.g., eye drops, is also within the skill in the art. The expression vector can also be administered non-invasively using a needleless injection device, such as the Biojector 2000 Needle-Free Injection Management System® available from Bioject, Inc.

The expression vector is preferably present in or on a device that allows controlled or sustained release of the expression vector, such as an ocular sponge, meshwork, mechanical reservoir, or mechanical implant. Implants (see, e.g., U.S. Pat. Nos. 5,443,505, 4,853,224 and 4,997,652), devices (see, e.g., U.S. Pat. Nos. 5,554,187, 4,863,457, 5,098,443 and 5,725,493), such as an implantable device, e.g., a mechanical reservoir, an intraocular device or an extraocular device with an intraocular conduit, or an implant or a device comprised of a polymeric composition are particularly useful for ocular administration of the expression vector. The expression vector of the present inventive methods can also be administered in the form of sustained-release formulations (see, e.g., U.S. Pat. No. 5,378,475) comprising, for example, gelatin, chondroitin sulfate, a polyphosphoester, such as bis-2-hydroxyethyl-terephthalate (BHET), or a polylacticglycolic acid.

Alternatively, the expression vector can be administered using invasive procedures, such as, for instance, intravitreal injection or subretinal injection, optionally preceded by a vitrectomy, or periocular (e.g., subtenon) delivery. The expression vector can be injected into different compartments of the eye, e.g., the vitreal cavity or anterior chamber. While intraocular injection is preferred, injectable compositions can also be administered intramuscularly, intravenously, intraarterially, and intraperitoneally. Pharmaceutically acceptable carriers for injectable compositions are well-known to those of ordinary skill in the art (see Pharmaceutics and Pharmacy Practice, J. B. Lippincott Co., Philadelphia, Pa., Banker and Chalmers, eds., pages 238-250 (1982), and ASHP Handbook on Injectable Drugs, Toissel, 4^{th} ed., pages 622-630 (1986)). Although less preferred, the expression vector can also be administered in vivo by particle bombardment, i.e., a gene gun.

Preferably, the expression vector is administered via an ophthalmologic instrument for delivery to a specific region of an eye. Use of a specialized ophthalmologic instrument ensures precise administration of the expression vector while minimizing damage to adjacent ocular tissue. Delivery of the expression vector to a specific region of the eye also limits exposure of unaffected cells to the inhibitor of angiogenesis and/or neurotrophic factor, thereby reducing the risk of side effects. A preferred ophthalmologic instrument is a combination of forceps and subretinal needle or sharp bent cannula.

While not particularly preferred, the expression vector can be administered parenterally. Preferably, any expression vector parenterally administered to a patient for the prophylactic or therapeutic treatment of an ocular-related disorder, i.e., ocular neovascularization or age-related macular degeneration, is specifically targeted to ocular cells. As discussed herein, an expression vector can be modified to alter the binding specificity or recognition of an expression vector for a receptor on a potential host cell. With respect to adenovirus, such manipulations can include deletion of regions of the fiber, penton, or hexon, insertions of various native or non-native ligands into portions of the coat protein, and the like. One of ordinary skill in the art will appreciate that parenteral administration can require large doses or multiple administrations to effectively deliver the expression vector to the appropriate host cells.

One of ordinary skill in the art will also appreciate that dosage and routes of administration can be selected to minimize loss of expression vector due to a host's immune system. For example, for contacting ocular cells in vivo, it can be advantageous to administer to a host a null expression vector (i.e., an expression vector not comprising the nucleic acid sequence encoding at least one inhibitor of angiogenesis and/or at least one neurotrophic factor) prior to performing the present inventive method. Prior administration of null expression vectors can serve to create an immunity (e.g., tolerance) in the host to the expression vector, thereby decreasing the amount of vector cleared by the immune system.

The dose of expression vector administered to an animal, particularly a human, in accordance with the invention should be sufficient to effect the desired response in the animal over a reasonable time frame. One skilled in the art will recognize that dosage will depend upon a variety of factors, including the age, species, the pathology in question, and condition or disease state. Dosage also depends on the inhibitor of angiogenesis and/or neurotrophic factor to be expressed, as well as the amount of ocular tissue about to be affected or actually affected by the ocular-related disease. The size of the dose also will be determined by the route, timing, and frequency of administration as well as the existence, nature, and extent of any adverse side effects that might accompany the administration of a particular expression vector and the desired physiological effect. It will be appreciated by one of ordinary skill in the art that various conditions or disease states, in particular, chronic conditions or disease states, may require prolonged treatment involving multiple administrations.

Suitable doses and dosage regimens can be determined by conventional range-finding techniques known to those of ordinary skill in the art. Preferably, about 10⁶ viral particles to about 10¹² viral particles are delivered to the patient. In other words, a pharmaceutical composition can be administered that comprises an expression vector concentration of from about 10⁶ particles/ml to about 10¹² particles/ml (including all integers within the range of about 10⁶ particles/ml to about 10¹² particles/ml), preferably from about 10¹⁰ particles/ml to about 10¹² particles/ml, and will typically involve the intraocular administration of from about 0.1 μl to about 100 μl of such a pharmaceutical composition per eye. In some instances, an injection can comprise from about 0.5 mL to about 1 mL of pharmaceutical composition. Ideally, a dose of about 1×10⁶, about 1×10^{6.5}, about 1×10⁷, about 1×10^{7.5}, about 1×10⁸, about 1×10^{8.5}, about 1×10⁹, about 1×10^{9.5} particles of adenoviral vector is administered per eye to a patient via intravitreal injection. Alternatively, the adenoviral vector of the inventive method is administered subretinally in a dose of about 1×10⁵, about 1×10^{5.5}, about 1×10⁶, about 1×10^{6.5}, about 1×10⁷, about 1×10^{7.5}, about 1×10⁸, or about 1×10^{8.5} particles per eye. When administered periocularly, the dose of adenoviral vector administered preferably is about 1×10⁷, about 1×10^{7.5}, about 1×10⁸, about 1×10^{8.5}, about 1×10⁹, about 1×10^{9.5}, about 1×10¹⁰, about 1×10^{10.5}, about 1×10¹¹, about 1×10^{11.5}, or about 1×10¹² particles per eye. When the expression vector is a plasmid, preferably about 0.5 ng to about 1000 μg of DNA is administered. More preferably, about 0.1 μg to about 500 μg is administered, even more preferably about 1 μg to about 100 μg of DNA is administered. Most preferably, about 50 μg of DNA is administered per eye. Of course, other routes of administration may require smaller or larger doses to achieve a therapeutic effect. Any necessary variations in dosages and routes of administration can be determined by the ordinarily skilled artisan using routine techniques known in the art.

In some embodiments, it is advantageous to administer two or more (i.e., multiple) doses of the expression vector comprising a nucleic acid sequence encoding at least one inhibitor of angiogenesis and/or at least one neurotrophic agent. The present inventive method provides for multiple applications of the inhibitor of angiogenesis and/or neurotrophic agent to prophylactically or therapeutically treat ocular neovascularization or other ocular-related disorders. For example, at least two applications of an expression vector comprising an exogenous nucleic acid, e.g., a nucleic acid sequence encoding at least one inhibitor of angiogenesis and/or at least one neurotrophic agent, can be administered to the same eye. Preferably, the multiple doses are administered while retaining gene expression above background levels. Also preferably, the ocular cell is contacted with two applications or more of the expression vector within about 30 days or more. More preferably, two or more applications are administered to ocular cells of the same eye within about 90 days or more. However, three, four, five, six, or more doses can be administered in any time frame (e.g., 2, 7, 10, 14, 21, 28, 35, 42, 49, 56, 63, 70, 77, 85 or more days between doses) so long as gene expression occurs and ocular neovascularization is inhibited or ameliorated. In a preferred embodiment, an adenoviral vector comprising a nucleic acid sequence encoding PEDF is administered to the same eye twice in three months or four times in six weeks.

Regulated expression of a therapeutic gene can be critical in affecting a biological response (e.g., a therapeutic response) in an animal. Long-term production or repeated-administration of a therapeutic factor can more efficiently treat progressive or chronic disorders of the eye (or any disorder or disease state regardless of location in the body) than a single bolus administration. However, in the context of adenovirus, expression of many genes under the direction of the CMV promoter has been reported to be transient in nature lasting approximately 2-4 weeks after administration into the vitreous of the eye and other locations in the body. Similar effects have been observed using other promoters in an adenoviral vector backbone. Additional or subsequent expression of therapeutic genes previously could only be achieved by repeatedly administering an expression vector to the target tissue (e.g., the eye or surrounding tissues). However, it has been surprisingly determined that transgene expression can be enhanced or upregulated after administering an adenoviral vector in vivo and can be re-activated after expression levels have waned. The invention provides a method of achieving long-term transgene expression, preferably a therapeutic transgene, without repeatedly administering an expression vector, e.g., an adenoviral vector. Long-term expression and, preferably, protein production is achieved by upregulating transcription of a transgene at any time point after administering an expression vector (e.g., an adenoviral vector), thereby re-activating protein production.

Thus, the invention provides a method of delivering a gene product to the eye. The method comprises (a) administering to an eye of an animal a first expression vector comprising a nucleic acid sequence operably linked to a promoter and encoding a gene product, such that the expression vector transduces an ocular cell (or multiple ocular cells) and the nucleic acid sequence is transcribed to produce the gene product. The method further comprises (b) upregulating transcription of the nucleic acid sequence in the ocular cell. Preferably, the expression vector is an adenoviral vector as described herein, and transcription is upregulated after the administration of the expression vector. Activation of expression is not limited to vector with a viral promoter (e.g., the CMV immediate early promoter) but also extends to the use of cellular promoters such as, for example, EF1-α (elongation factor 1-α), which is composed, at least in part, of jun and fos, the Ubiquitin C (UbC) promoter, and the Ying Yang 1 (YY1) promoter.

The method of upregulating transcription is particularly useful in providing therapeutic factors, such as the therapeutic factors described herein, to the eye to treat an ocular-related disorder. The method of treating an ocular-related disorder comprises (a) administering to an animal a first expression vector comprising a nucleic acid sequence encoding an inhibitor of angiogenesis and/or a neurotrophic agent such that the expression vector transduces at least one ocular cell and the nucleic acid sequence is transcribed. The method further comprises (b) upregulating transcription of the nucleic acid sequence. Expression of the inhibitor of angiogenesis and/or a neurotrophic agent is thereby upregulated to prophylactically or therapeutically treat the animal for an ocular-related disorder.

The upregulation of transcription can result in increased levels of RNA transcript, increased protein production, and/or an enhancement in detectable gene product activity, all of which can be detected using routine laboratory techniques. Transcription is upregulated in the ocular cell by altering the environment of the cell by, for example, administering exogenous materials to the eye and/or inducing a stress response in the eye. Exogenous material can be administered directly to the eye (which in come cases induces a stress response in the eye) or can be administered at a site other than the eye. Exemplary routes of administration are described herein and include, for example, topical, subconjunctival, retrobulbar, periocular, subtenon, subretinal, suprachoroidal, or intraocular administration. For example, periocular injection allows delivery of proteins and/or nucleic acids to the retina. Thus, a sustained release device can be implanted in the periocular space to administer substances to various regions of the eye. Administering the exogenous substance orally, intravenously, intraarterially, intramuscularly, subcutaneously, intraperitoneally, parenterally, intranasally, transdermally, systemically, or intratracheally also is appropriate. The exogenous material(s) can be formulated for any suitable route of administration. For example, an exogenous material can be formulated into eye drops, ointment for topical delivery to the eye, composition for oral delivery, or parenteral solution for systemic delivery of, for example, a retinoic acid (e.g., all trans-retinoic acid, 9-cis-retinoic acid, NPB, or LG100064).

Exogenous materials suitable for administering to the animal to upregulated transcription of a nucleic acid sequence in the eye include, but are not limited to, any of the substances described herein such as saline, a disaccharide, such as trehalose, a protein, a nucleic acid, and a drug (e.g., phorbolesters and the like). If administering a protein, the protein is preferably a cytokine, an inhibitor of angiogenesis (e.g., soluble flt (s-flt) or pigment epithelium-derived factor (PEDF)), a neurotrophic agent, a steroid, an enzyme (e.g., hyaluronidase), or an antibody (e.g., an anti-VEGF antibody). If administering a nucleic acid, preferably the nucleic acid is an aptarner, siRNA, or double-stranded RNA. Suitable drugs for upregulating transcription include, but are not limited to, an immunosuppressant (e.g., cyclosporine, a glucocorticoid, or SK506), a steroid derivative, diclofenac sodium and misoprostol, dixlurenac, combretastatin, a protein kinase C (PKC) inhibitor (e.g., LY333531 (see Danis et al., Invest. Ophthalmol. Vis. Sci., 39(1), 171-9 (1998))), a tyrosine kinase (TK) inhibitor (Seo et al., Am. J Pathol., 154(6), 1743-53 (1999)), a Cox-I inhibitor, a Cox-II inhibitor (e.g., nepafenac), an anti-inflammatory agent, aspirin, or hyaluronic acid. Alternatively or additionally, a second expression vector (e.g., a second adenoviral vector) can be administered to the animal. Ideally, the second adenoviral vector is deficient in all replication-essential gene finctions encoded by the E4 region of the adenoviral genome. More preferably, the adenoviral vector is deficient in all gene finctions of the E4 region of the adenoviral genome. The second adenoviral vector ideally does not comprise the nucleic acid sequence present in the first expression (e.g., adenoviral) vector. The second expression vector need not encode a therapeutic protein.

Preferred compounds to administer to upregulate transcription are histone deacetylase inhibitors, which can have anti-angiogenic and anti-cancer activity, and a retinoic acid. The histone deacetylase inhibitor can inhibit any mammalian Class I, Class II, or Class HI histone deacetylase enzyme including, but not limited to, HDAC1 and HDAC2, HDAC3, HDAC8, HDAC1 1, HDAC4 and HDAC5, HDAC6, HDAC7, HDAC9, HDAC10, or the Sirtuins. Exemplary histone deacetylase inhibitors include, for example, short-chain fatty acids, butyrate and phenylbutyrate, valproate, hydroxamic acids, trichostatins, SAHA and derivatives thereof, oxamflatin, ABHA, scriptaid, pyroxamide, propenamides, epoxyketone-containing cyclic tetrapeptides, trapoxins, HC-toxin, chlamydocin, diheteropeptin, WF-3161, Cyl-1 and Cyl-2, non-epoxyketone-containing cyclic tetrapeptides, FR901228, apicidin, cyclic-hydroxamic-acid-containing peptides (CHAPs), benzamides and analogues thereof, MS-275 (MS-27-275), CI-994, depudecin, and organosulfur compounds. Likewise, a variety of functional analogues of retinoic acid are known in the art, such as all trans-retinoic acid, 9-cis-retinoic acid, NPB, and LG100064. Retinoic acid binds at least one of two families of retinoic acid receptors, RARs and RXRs. Upon binding of retinoic acid to a retinoic acid receptor, the retinoic acid receptor can dimerize, thereby forming active receptor complexes which interact with retinoic acid responsive elements.

In a preferred embodiment, the exogenous material administered to the animal is not a pyrogen, such as lipopolysaccharide, which can cause inflammation in many tissues. Thus, the method of the invention desirably comprises administering a non-pyrogen activator of transcription (such as those exogenous materials described herein). Likewise, in some embodiments, it is preferable not to administer an adenoviral vector (or other expression vector) to upregulate transcription. Repeated administration of an adenoviral vector can cause inflammation, particularly in the eye. Exogenous material derived from adenovirus also can cause inflammation, and may not be suitable for upregulating transcription in some instances. Radiation also can cause tissue damage, and may not be preferred in some instances. It will be appreciated that exogenous materials which cause inflammation, an immune response, or damage transduced cells will not be appropriate in many instances, in particular those situations wherein cell protection is desired. In addition, transcription re-activation can be achieved at any time point so long as the expression vector is present. Elimination of transduced cells is not desired in this respect.

Transcription also can be upregulated by inducing a stress response in the eye. A stress response can be induced by piercing the eye, exposure to heat using, for example, lasers in photodynamic therapy, exposure to cold, exposure to light, exposure to radiation (e.g., X-rays), exposure to microwaves, exposure to ultrasound, or physical trauma, all of which can alter the ocular cellular environment to enhance transcription. An alternative method of altering an ocular cell environment is by administering a puff of air to the eye, as is commonly administered during glaucoma testing. Alternatively, the stress response can be induced by administering an exogenous substance, such as a nucleic acid, a lipid, a drug, and others described herein, or any combination of the foregoing that induces a stress response, or itself is an active participant in a cellular stress response.

The ability to re-activate transcription from an adenoviral vector is not limited to the eye. Indeed, the invention provides a method of delivering a gene product to a mammal. The method comprises (a) administering to the mammal an adenoviral vector deficient in all replication-essential gene functions of the E4 region of the adenoviral genome and comprising a nucleic acid sequence operably linked to a promoter and encoding a gene product, such that the adenoviral vector transduces a host cell and the nucleic acid sequence is transcribed to produce a gene product. The method further comprises (b) subsequently upregulating transcription of the nucleic acid sequence in the host cell. In the method, upregulating transcription does not comprise administering a pyrogen, an adenoviral vector, or radiation. Upregulating transcription can comprise exposing the host cell to the same stimuli as described above with respect to ocular cells. For instance, transcription can be upregulated by exposing one or more transduced host cells to an exogenous material that upregulates transcription, cold, light, microwaves, ultrasound, or physical trauma.

Transcription can be upregulated as determined by the expression profile desired by the practitioner. Ideally, the method comprises upregulating transcription after administering the first expression vector. In that the viral genome is stable in the eye (˜20% of day one level detected for at least 1 month post-administration) and is maintained for a year or more, adenoviral vectors provide a means of delivering proteins over extended periods of time without repeated dosing. While the practitioner can alter the cellular environment simultaneously with the administration of the expression vector (e.g., adenoviral vector) to enhance and upregulate initial expression levels, preferably, transcription is upregulated subsequent to expression vector administration. Transcription can be upregulated multiple (i.e., two or more) times from the same adenoviral backbone. Most promoters lose activity after a period of time (e.g., two weeks). In one aspect, the transcription is upregulated in response to loss of promoter activity in order to re-activate expression of transgene products. Transcription is preferably upregulated at least once within one day of administering the adenoviral vector, more preferably at least once within seven days of administering the adenoviral vector (e.g., at least once within 14 days of administering the adenoviral vector). Ideally, transcription is upregulated at least once within 21 days, preferably at least once with 28 days, of administering the adenoviral vector. Alternatively, transcription is upregulated at least once within 35 days, 42 days, or 48 days of administering the adenoviral vector. More preferably, transcription is upregulated at least once within three months (e.g. four months or five months), even more preferably upregulated at least once within six months (e.g., seven months, eight months, nine months or more) of administering the adenoviral vector. Most preferably, transcription is upregulated at least once within 12 months (i.e., 1 year) of administering the adenoviral vector. Depending on the lifespan of the animal, expression can be re-activated after many years if desired (i.e., 10 or 20 years). Indeed, transcription can be upregulated or re-activated so long as the expression vector is present in the host cell (and the transduced host cell is functional). According, transcription can be re-activated or upregulated as needed for about 1, 2, 3, 4, 5, 6, 7, 8, 9, or 10 years or more following administration of the expression vector depending on the lifespan of the transduced host cell(s).

The time between administering the first expression vector (e.g., adenoviral vector) and upregulating transcription of the encoded transgene (e.g., steps (a) and (b) of the inventive method) can be determined by the practitioner on a case-by-case basis. Desirably, the time between administering the first expression vector (e.g., adenoviral vector) and upregulating transcription is at least one day (e.g., at least four days, at least seven days, or at least 14 days). Alternately, the time between administering the first expression vector (e.g., adenoviral vector) and upregulating transcription is at least 28 days (e.g., at least 48 days, at least 60 days, or at least 3 months). In addition, the time between administering the first expression vector (e.g., adenoviral vector) and upregulating transcription can be at least 6 months (e.g., at least 9 months or 1 year). In one embodiment, transcription is upregulated after initial transcription levels have diminished 2-, 5-, or 10-fold.

In addition, transcription can be upregulated or re-activated any number of times after administration of the expression vector. Transcription can be upregulated or re-activated, for example, one time to about 50 times (e.g., 1, 2, 3, 4, 5, 6, 7, 8, 9, 10, 11, 12, 13, 14, 15, 20, 25, 30, 40, or 50 times).

Upregulating (re-activating) transcription of a nucleic acid sequence by the inventive method results in an increase in transcription relative to the level of transcription of the nucleic acid sequence absent the upregulation transcription at the time point tested (i.e., the same timepoint on an expression profile). Preferably, the level of transcription following upregulation of transcription is greater than the level of transcription of the nucleic acid sequence absent the upregulation of transcription, no matter at what time point in the expression profile the peak level of transcription occurs. Surprisingly, the expression competence of an adenoviral vector construct was retained for at least 28 days and could be upregulated by about 10- to about 100-fold using the inventive method. Accordingly, the level of transcription of the nucleic acid sequence of the inventive method is preferably enhanced at least about 2-fold compared to the level of transcription of the nucleic acid sequence absent the upregulation of transcription. More preferably, the level of transcription of the nucleic acid sequence is greater than at least about 5-fold (e.g., at least about 10-fold, about 20-fold, about 25-fold, about 35-fold, about 40-fold, or about 45-fold) greater than the level of transcription of the nucleic acid sequence absent the upregulation of transcription. Even more preferably, the level of transcription of the nucleic acid sequence is at least about 50-fold (e.g., at least about 55-fold, about 60-fold, about 65-fold, or about 70-fold) greater than the level of transcription of the nucleic acid sequence absent the upregulation of transcription. Most preferably, the level of transcription of the nucleic acid sequence is at least about 75-fold (e.g., at least about 80-fold, about 85-fold, about 90-fold, about 95-fold, or about 100-fold) greater than the level of transcription of the nucleic acid sequence absent the upregulation of transcription.

On the other hand, the level of transcription achieved following upregulation can be compared to the level of transcription at one day following administration of the first expression vector (e.g., adenoviral vector). In some instances, the level of expression at one day post-administration of expression vector is the peak level of transcription. Preferably, the level of transcription at one day following transcription upregulation is at least about 20% (e.g., at least about 25%, at least about 35%, or at least about 45%) the level of transcription of the nucleic acid sequence at one day post-administration of the first expression vector (e.g., adenoviral vector). More preferably, the level of transcription at one day following transcription upregulation is at least about 50% (e.g., at least about 60%, at least about 70%, at least about 80%, or at least about 90%) the level of transcription of the nucleic acid sequence at one day post-administration of the first expression vector (e.g., adenoviral vector). Most preferably, the level of transcription at one day following transcription upregulation is at least about 100% (e.g., more than 100%) the level of transcription of the nucleic acid sequence at one day post-administration of the first expression vector (e.g., adenoviral vector).

In one embodiment, the inventive method of delivering a gene product to a mammal comprises (a) administering to the mammal (i) an expression vector comprising a first nucleic acid sequence operably linked to a promoter such that the expression vector transduces a host cell and the nucleic acid sequence is transcribed to produce a gene product and (ii) a second nucleic acid sequence operably linked to a promoter and encoding a retinoic acid receptor, wherein the second nucleic acid sequence is transcribed in the host cell to produce a retinoic acid receptor. The method further comprises (b) subsequently administering to the mammal a retinoic acid, thereby upregulating transcription of the first nucleic acid in the host cell. The second nucleic acid sequence can be present in the same expression vector as the first nucleic acid sequence, or can be provided on a different (i.e., second) expression vector. Ideally the second nucleic acid sequence is co-administered with the first nucleic acid sequence, but simultaneous administration is not required. The second nucleic acid sequence can be administered before, during, or after administration of the first nucleic acid sequence, so long as the second nucleic acid sequence is not administered after step (b). By exogenously producing a retinoic acid receptor, transcription can be upregulated using retinoic acid in cells that do not express a retinoic acid receptor or cells that do not express enough retinoic acid receptors to achieve the desired level of transcription upregulation.

It also will be appreciated by one skilled in the art that an expression vector comprising a nucleic acid sequence encoding at least one inhibitor of angiogenesis and/or at least one neurotrophic factor can be introduced ex vivo into cells, preferably ocular cells, previously removed from a given animal, in particular a human. Such transduced autologous or homologous host cells, reintroduced into the animal or human, will express directly at least one inhibitor of angiogenesis and/or at least one neurotrophic factor in vivo. One ex vivo therapeutic option involves the encapsidation of infected ocular cells into a biocompatible capsule, which can be implanted in the eye or any other part of the body. One of ordinary skill in the art will understand that such cells need not be isolated from the patient, but can instead be isolated from another individual and implanted into the patient.

It will be appreciated that the expression vector, preferably the adenoviral vector, can comprise multiple nucleic acid sequences encoding the at least one inhibitor of angiogenesis and/or the at least one neurotrophic factor. For example, the expression vector can comprise multiple copies of the PEDF coding sequence, each copy operably linked to a different promoter or to identical promoters.

In addition to the above, the nucleic acid sequence encoding at least one inhibitor of angiogenesis and/or at least one neurotrophic factor can further comprise one or more other transgenes. By “transgene” is meant any nucleic acid that can be expressed in a cell. Desirably, the expression of the transgene is beneficial, e.g., prophylactically or therapeutically beneficial, to the ocular cell or eye. If the transgene confers a prophylactic or therapeutic benefit to the cell, the transgene can exert its effect at the level of RNA or protein. For example, the transgene can encode a peptide other than an inhibitor of angiogenesis or neurotrophic factor that can be employed in the treatment or study of a disorder, e.g., an ocular-related disorder. Alternatively, the transgene can encode an antisense molecule, a ribozyme, siRNA, a protein that affects splicing or 3′ processing (e.g., polyadenylation), or a protein that affects the level of expression of another gene within the cell (i.e., where gene expression is broadly considered to include all steps from initiation of transcription through production of a process protein), such as by mediating an altered rate of mRNA accumulation or transport or an alteration in post-transcriptional regulation. The transgene can encode a chimeric peptide for combination treatment of an ocular-related disorder. The transgene can encode a factor that acts upon a different target molecule than the inhibitor of angiogenesis or the neurotrophic agent. Indeed, the transgene product can act upon a different signal transduction pathway, or can act at different points of the same signal transduction pathway of the inhibitor of angiogenesis or the neurotrophic factor. Preferably, the therapeutic substance is a neurotrophic factor, such as CNTF. CNTF belongs to the neuropoietic cytokines subclass of neurotrophic factors. CNTF promotes the survival of ciliary ganglionic neurons and supports certain neurons that are nerve growth factor (NGF)-responsive. Likewise, a nucleic acid sequence encoding a receptor for an exogenous material for upregulating transcription (e.g., a retinoic acid receptor) can be included on the expression vector or on a different expression vector administered to the patient.

Alternatively, one or more additional nucleic acid sequences (e.g., transgenes) that encode a factor associated with cell differentiation can be included in the expression vector (e.g., viral vector). Preferably, the transgene encodes an atonal-associated peptide such as Math1 or Hath1 or a biologically active fragment of either of the foregoing. Math1 is a member of the mouse basic helix-loop-helix family of transcription factors and is homologous to the Drosophila gene atonal. HathI is the human counterpart of Math1. Math1 has been shown to be essential for hair development and can stimulate hair regeneration in the ear. Combining neurotrophic activity and the hair cell differentiation properties of an atonal-associated peptide provides a powerful tool for the treatment and research of, for example, sensory disorders. Math1 is further characterized in, for example, Bermingham et al., Science, 284, 1837-1841 (1999) and Zheng and Gao, Nature Neuroscience, 3(2), 580-586 (2000).

The expression vector can comprise a nucleic acid sequence encoding a vessel maturation factor in addition to at least one inhibitor of angiogenesis and/or at least one neurotrophic factor. Many ocular disorders involve leakage of blood products through vessels, which can cloud vision and induce an immune response within the layers of the eye. Vessel maturation factors reduce the amount of vascular leakage and, therefore, are useful in treating, for example, exudative ocular disorders. Vessel maturation factors include, but are not limited to, angiopoietins (Ang, e.g., Ang-1 and Ang-2), tumor necrosis factor-alpha (TNF-α), midkine (MK), COUP-TFII, hepatic growth factor (HGF), and heparin-binding neurotrophic factor (HBNF, also known as heparin binding growth factor). A nucleotide sequence encoding an immunosuppressor also can be incorporated into the expression vector to reduce any inappropriate immune response within the eye as a result of an ocular-related disorder or the administration of the expression vector.

The expression vector can comprise one or more additional nucleic acid sequences encoding an additional anti-angiogenic substance. As set forth above, an anti-angiogenic substance is any biological factor that prevents or ameliorates neovascularization. One of ordinary skill in the art will understand that the anti-angiogenic substance can effect partial or complete prevention and amelioration of angiogenesis to achieve a therapeutic effect. An anti-angiogenic substance includes, for instance, an anti-angiogenic factor, an anti-sense molecule specific for an angiogenic factor, a ribozyme, siRNA, a receptor for an angiogenic factor, and an antibody that binds a receptor for an angiogenic factor. Suitable anti-angiogenic factors include, but are not limited to, combretastatin, which is an anti-tubule factor, and endostatin.

The transgene can encode a marker protein, such as green fluorescent protein or luciferase. Such marker proteins are useful in vector construction and determining vector migration. Marker proteins also can be used to determine points of injection or treated ocular tissues in order to efficiently space injections of the expression vector to provide a widespread area of treatment, if desired. Alternatively, the transgene can encode a selection factor, which also is useful in vector construction protocols. If desired, the transgene can be part of an expression cassette.

It should be appreciated that any of the nucleic acid sequences described herein can be altered from their native form to increase their therapeutic effect. For example, a cytoplasmic form of a therapeutic nucleic acid can be converted to a secreted form by incorporating a signal peptide into the encoded gene product. The at least one inhibitor of angiogenesis and/or at least one neurotrophic factor can be designed to be taken up by neighboring cells by fusion of the peptide with VP22. This allows an ocular cell comprising the therapeutic nucleic acid to have a therapeutic effect on a number of ocular cells within the mammal.

The present inventive methods also can involve the co-administration of other pharmaceutically active compounds. By “co-administration” is meant administration before, concurrently with, e.g., in combination with the expression vector in the same formulation or in separate formulations, or after administration of the expression vector as described above. Any of the exogenous materials, drugs, proteins, and the like described herein can be co-administered with the expression vector as adjuvant therapy. For example, factors that control inflammation, such as ibuprofen or steroids, can be co-administered to reduce swelling and inflammation associated with intraocular administration of the expression vector. Immunosuppressive agents can be co-administered to reduce inappropriate immune responses related to an ocular disorder or the practice of the present inventive method. Anti-angiogenic factors, such as soluble growth factor receptors, growth factor antagonists, i.e., angiotensin, and the like can also be co-administered, as well as neurotrophic factors. In addition, the expression vector of the inventive method can be administered with anti-proliferative agents such as siRNA, aptamers, or antibodies which sequester or inactivate angiogenic factors such as, for example, VEGF. Similarly, vitamins and minerals, anti-oxidants, and micronutrients can be co-administered. Antibiotics, i.e., microbicides and fungicides, can be co-administered to reduce the risk of infection associated with ocular procedures and some ocular-related disorders. Other therapeutics for ocular disorders can be administered in conjunction with the inventive method. For example, Visudyne® (Novartis), Macugen™ (Pfizer), Retaane™ (Alcon), Lucentis™ (Genentech/Novartis), Squalamine (Genaera), Cosopt, and Alphagan can be formulated with the first expression vector or can be administered separately before, during, or after administration of the first expression vector to the animal.

Although the expression vectors of the instant invention are particularly useful in the study or treatment of ocular disorders, including ocular disorders comprising a neovascular component and age-related macular degeneration, it will be appreciated that the expression vectors can be used to research and/or treat prophylactically or therapeutically a wide variety of animal diseases. For example, the viral vector comprising a nucleic acid sequence encoding PEDF or a therapeutic fragment thereof can be used in the study or treatment of the nervous system, genitourinary ailments, cancer, infectious disease, and cardiovascular abnormalities, as well as miscellaneous other health nuisances. The viral vector can be used to study or treat, for example, sleep disorders, ALS (Lou Gehrig's Disease), Alzheimer's Disease, epilepsy, multiple sclerosis, Parkinson's Disease, peripheral neuropathies, Schizophrenia, depression, anxiety, spinal cord injury, traumatic brain injury, or acute, chronic, or inflammatory pain. The expression vectors of the invention can be used to treat genitourinary ailments, which include, for example, benign prostatic hyperplasia (BPH), impotence, neurogenic bladder, urinary incontinence, kidney failure, and end stage renal disease. The expression vectors are useful in treating cancer such as, for example, cancer of the bladder, brain, breast, colorectal, esophageal, head & neck, liver/hepatoma, lung, melanoma, ovarian, pancreatic, prostate, stomach, testicular, uterine/endometrial, leukemias, and lymphomas. Exemplary infectious diseases for treatment with the expression vectors include, but are not limited to, chlamydia, herpes, malaria, human papilloma virus (HPV), AIDS/HIV, pneumococcal pneumonia, influenza, meningitis, hepatitis, and tuberculosis. Cardiovascular diseases such as, for example, neovascular diseases, ischemia, congestive heart failure, coronary artery disease, arrhythmia, atherosclerosis, increased LDL/HDL ratios, restenosis after angioplasty or instent restenosis, stroke, sickle cell anemia, and hemophilia, can be treated or studied, as well as the alleviation of, for example, obesity, organ transplantation/transplant rejection, osteoporosis, alopecia, hair loss, arthritis, allergies (such as to ragweed, pollen, and animal dander), cystic fibrosis, diabetes, and hearing loss. One of ordinary skill in the art will appreciate that animal models exist for many of the disease states identified above.

### EXAMPLES

The following examples further illustrate the invention but, of course, should not be construed as in any way limiting its scope.

### Example 1

This example illustrates a preferred method of obtaining expression of a factor comprising both anti-angiogenic and neurotrophic activity from an adenoviral vector in in vivo retina.

An adenoviral vector deficient in one or more essential gene functions of the E1, E3, and E4 regions of the adenoviral genome and comprising a PEDF gene (Ad.PEDF) is preferably constructed as set forth in WO 99/15686 (McVey et al.). However, the method of the invention is not dependent on the method of vector construction employed and previously described methods of vector construction are also suitable.

Several in vivo models of ocular neovascularization are available. Neovascularization of the retina is obtained in, for example, neonatal animals, i.e., neonatal mice, by exposing the mice to hypoxic conditions shortly after birth. Several days later, the neonatal mice are exposed to standard atmospheric conditions, resulting in ischemia-induced neovascularization of the retina.

Ad.PEDF is administered to the right eye of at least 12 day old mice anesthetized with, for example, ketamine or a combination of ketamine and xylazine via intravitreal injection. Injections are performed by forming an entrance site in the posterior portion of the eye and administering approximately 0.1-5.0 μl of composition comprising Ad.PEDF. In most instances, an injection of the expression vector will be administered to only one eye, while the remaining eye serves as a control. The mice are sacrificed at various time points after administration to determine the extent and duration of PEDF expression in the retina. The right and left eyes of each animal are enucleated and either fixed for histological analysis or prepared for PEDF expression analysis. Detection of PEDF DNA, PEDF RNA, or PEDF protein can be accomplished using methods well known in the art, such as PCR and blotting techniques (see, for example, Sambrook et al., supra).

To determine the effect of PEDF on neovascularization in vivo in, for example, a human, indirect ophthalmoscopy of the retina is ideal. Stereophotographs are useful in detecting extensive neovascularization, but not appropriate for detecting subtle lesions.

### Example 2

This example demonstrates a preferred method of obtaining expression of a factor comprising both anti-angiogenic and neurotrophic activity from an adenoviral vector in in vivo choroid. The following example further provides methods for determining the effect of PEDF on neovascularization.

An adenoviral vector deficient in one or more essential gene functions of the E1, E3, and E4 regions of the adenoviral genome and comprising a PEDF gene (Ad.PEDF) is constructed as set forth in WO 99/15686 (McVey et al.).

An in vivo model of choroidal neovascularization can be obtained by detaching the retina of an eye of, for example, a mouse or rabbit, and debriding the pigmented epithelia. Choriocapillary regeneration is monitored in both treated and untreated eyes. Ad.PEDF is administered prior to perturbing the retinal pigment epithelial (RPE) to determine the effect of the present inventive method in preventing choroidal neovascularization. Of course, Ad.PEDF is administered after perturbing the retina and RPE for determining the therapeutic effect of the procedure on neovascularization.

Choroidal neovascularization can be monitored in vivo using flndus photography, fluorescein angiography and/or indocyanine-green angiography, as commonly used in the art. Using these methods, one of ordinary skill in the art is able to detect growth of new blood vessels and vascular leakage often associated with neovascularization. For research purposes, neovascularization can also be determined by enucleating the eyes and preparing vascular casts or examining ocular tissue via scanning electron microscopy.

### Example 3

This example demonstrates the utility of adenoviral vectors to deliver multiple doses of an exogenous nucleic acid to the eye.

Adenoviral vectors comprising the luciferase gene (Ad.L) or control adenoviral vectors comprising no transgene (Ad.null) were injected into the intravitreal space of C57BL6 mouse eyes (Day 0). One day following injection of the adenoviral vectors (Day 1), eyes infected with Ad.L were enucleated and frozen (1^{st} administration). The eyes infected with Ad.null were divided into three groups. In Group I, Ad.L vectors were injected into the intravitreal space of the eye at Day 14 (fourteen days following the initial dose of Ad.null). Group I eyes were enucleated and frozen the day following the second administration of adenoviral vectors (Day 15, 2^{nd} administration). Group II eyes were injected intravitreally with Ad.null at Day 14, and injected intravitreally with Ad.L vectors four weeks following the initial injection with Ad.null (Day 28, 3^{rd} administration). The eyes were then enucleated and frozen the day after the third administration of adenoviral vector. Group III eyes were injected intravitreally with Ad.null at Day 14 and Day 28, and injected with Ad.L vectors six weeks following the initial injection with Ad.null (Day 42, 4^{th} administration). The eyes were then enucleated and frozen the day after the fourth administration of adenoviral vector. Luciferase assays were performed on the eye samples to determine the efficiency of infection and gene expression associated with multiple dosing of the vectors.

Luciferase expression in ocular cells after the 1^{st} and 2^{nd} administration of adenoviral vector was substantially equivalent. In other words, no loss of gene expression was detected following two administrations of the gene transfer vector. Gene expression from the 3^{rd} administration of adenoviral vector was between 10- and 100-fold reduced compared to gene expression from the 1^{st} administration and the 2^{nd} administration, but was still above background levels (e.g., as detected in cells transduced with Ad.null). Gene expression from the 4^{th} administration of adenoviral vector was reduced approximately 3- to 10-fold compared to the gene expression observed following the 3^{rd} administration. However, the level of gene expression following the 4^{th} administration was above background levels.

This example demonstrates the feasibility of performing multiple applications of adenoviral vectors to the eye in order to obtain expression of an exogenous nucleic acid in ocular cells.

### Example 4

This example demonstrates the ability of an expression vector comprising a nucleic acid sequence encoding a factor comprising both anti-angiogenic and neurotrophic properties to inhibit choroidal neovascularization (CNV).

Replication-deficient (E1-/E3-deficient) adenoviral vectors (AdPEDF.10) comprising the coding sequence for PEDF operably linked to the CMV immediate early promoter were constructed using standard techniques. A null version of the vector (AdNull.10), which did not comprise the PEDF coding sequence, was also constructed.

Adult C57BL/6 mice were injected intravitreously with AdNull.10 or AdPEDF.10 using a Harvard pump microinjecfion apparatus and pulled glass micropipettes. Each eye was injected intravitreously with 1 μl of vehicle containing 10⁹ particles of virus. Alternatively, each eye was injected subretinally with 10⁸ particles of virus suspended in 1 μl of vehicle. Five days post-injection, mice were anesthetized with ketamine hydrochloride (100 mg/kg body weight). Topicamide (1%) was utilized to dilate the pupils prior to rupture of Bruch's membrane by diode laser photocoagulation. Rupture of Bruch's membrane is known to induce neovascularization of the choroid.

Fourteen days following laser-induced rupture of Bruch's membrane, choroidal flat mounts (described in Edelman et al., Invest. Ophthalmol. Vis. Sci., 41, S834 (2000)) were prepared to observe the degree of neovascularization of the choroidal membrane. Briefly, eyes were removed from the subjects and fixed in phosphate-buffered formalin. The cornea, lens, and retina were removed from the eyecup, and the eyecup was flat-mounted. Flat mounts were then examined by fluorescence microscopy and images were digitized using a 3 color CCD video camera (IK-TU40A, Toshiba, Tokyo, Japan) for computer image analysis.

Large areas of neovascularization were observed in uninjected eyes and eyes receiving AdNull.10. Eyes injected with AdPEDF.10 subretinally or intravitreously showed smaller regions of neovascularization compared to the controls using computerized image analysis.

The above results illustrate the ability of the present inventive method to inhibit ocular neovascularization, namely choroidal neovascularization (CNV), in a clinically animal relevant model.

### Example 5

This example demonstrates the ability of an expression vector comprising a nucleic acid sequence encoding a factor comprising both anti-angiogenic and neurotrophic properties to inhibit ischemia-induced retinal neovascularization.

Replication-deficient adenoviral vectors comprising the coding sequence for PEDF operably linked to the CMV immediate early promoter were constructed using standard techniques. E1-/E3-/E4-deficient vectors encoding PEDF (AdPEDF.11) and a null version of the vector (AdNull.11), which did not comprise the PEDF coding sequence, were constructed.

Ischemic retinopathy was produced in adult C57BL/6 mice as previously described (see, for example, Smith et al., Invest. Ophthalmol. Vis. Sci., 35, 101 (1994)). Briefly, seven day old mice (P7) were exposed to an atmosphere of 75+/−3% oxygen for five days. At P10, mice were injected intravitreously with 10⁹ particles of AdPEDF.11 or AdNull.11, returned to oxygen for two days, then returned to room atmosphere. At P17, the mice were sacrificed and eyes were rapidly removed and frozen in optimum cutting temperature embedding compound (OCT; Miles Diagnostics, Elkhart, Ind.).

To detect neovascularization, the eyes were sectioned and histochemically stained with biotinylated griffonia simplicifolia lectin B4 (GSA, Vector Laboratories, Burlingame, Calif.). Slides were then incubated in methanol/H₂O₂ for 10 minutes at 4° C., washed with 0.05 M Tris-buffered saline, pH 7.6 (TBS), and incubated for 30 minutes in 10% normal porcine serum. The slides were then incubated for two hours with biotinylated GSA, rinsed with TBS, and incubated with avidin-coupled alkaline phosphatase (Vector Laboratories) for 45 minutes. After a 10 minute wash with TBS, the slides were incubated with Histomark Red. GSA-stained, 10 μm serial sections were examined using an Axioskop microscope. Images were digitized using a 3 color CCD video camera (IK-TU40A, Toshiba, Tokyo, Japan) for computer image analysis.

Extensive retinal neovascularization was detected in eyes not injected with any virus. Eyes injected with AdNull.11 showed less neovascularization than uninjected eyes, but significantly more neovascularization of the retina than eyes injected with AdPEDF.11. Eyes injected with AdPEDF.11 comprised the least amount of neovascularization.

This example clearly demonstrates the ability of the present inventive method to inhibit an ocular-related disorder, namely ischemia-induced retinal neovascularization, in a clinically relevant animal model.

### Example 6

This example demonstrates that adenoviral vector genomes remain in the eye for at least 28 days.

Expression of many genes under the direction of the CMV promoter in an adenoviral vector has been reported to be transient in nature lasting approximately 2 weeks after administration into the vitreous of the eye. The loss of expression could be due to the clearance of vector genomes from the eye or the shut off of expression from the vector genomes. To address these possible mechanisms, the presence of vector genomes in the eye was measured after intravitreal delivery of the adenoviral vector. Replication-deficient adenovirus deleted of E1, E4, and E3 (partially) adenoviral early gene regions was delivered into the eyes of mice via intravitreal injection as described in Example 5. The amount of vector genome was quantitated using a sensitive and specific quantitative PCR assay. A dose response for the genome in vitro and in vivo showed the sensitivity and reliability of the qPCR assay. The amount of adenoviral vector genome in the eye was quantitated as a function of dose and time post administration. The level of vector genome in the eye at one day post-administration correlated directly with the amount of vector particles administered. These data showed the amount of vector genomes remained remarkably constant after 28 days post administration while expression dropped rapidly.

This example suggests that the transient nature of expression from adenovirus vectors is due to expression shut off and not loss of adenoviral vector genomes from ocular tissue. The amount of adenoviral vector genomes remained constant for at least 28 days post-intravitreal injection.

### Example 7

This example demonstrates the modulation of transgene expression from an adenoviral vector by altering the cellular environment by inducing a stress response in a host cell.

A time course of expression of a marker gene, namely the luciferase gene, delivered to the vitreous cavity of the eye as part of an adenoviral serotype 5 vector was determined. The adenoviral vector genome was deficient in one or more essential gene functions of the E1, E3, and E4 regions of the adenoviral genome and comprised the luciferase gene (AdL.11D). The luciferase gene under the control of the CMV immediate early promoter replaced the E1 region of the adenoviral genome while the E4 region was replaced with a spacer sequence that is not transcribed.

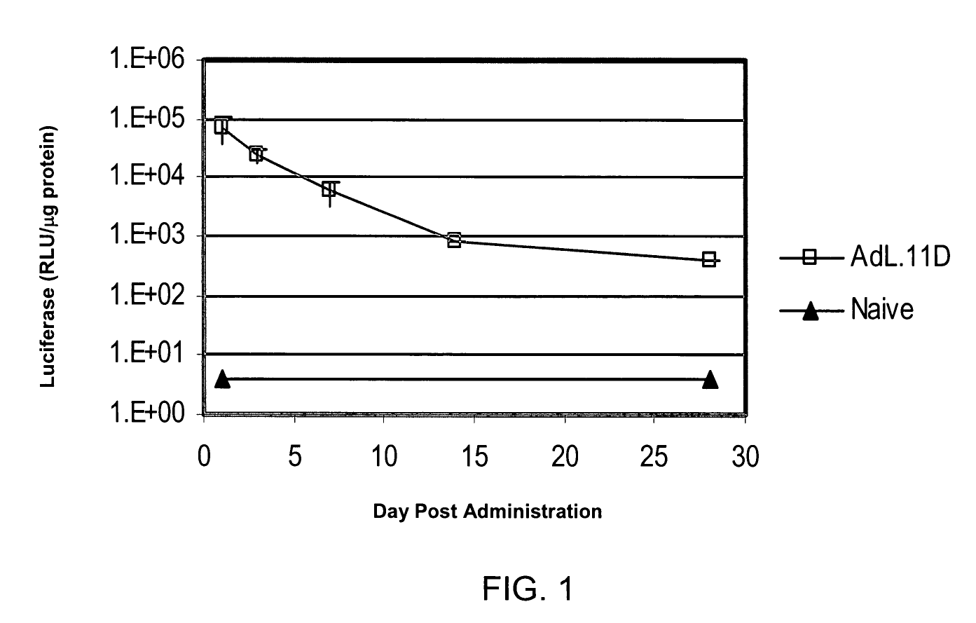

A total of 1×10⁷ particle units (pu) were injected intravitreously into C57BL/6 mice. The eyes were harvested at various days post administration and relative levels of luciferase activity was determined. Measurement of luciferase activity is an accepted method of studying transcription. An initial burst of expression was observed on day 1 post-administration that decreased by 7-10 fold by week two as depicted in FIG. 1. This lower level of expression remains above the background signal.

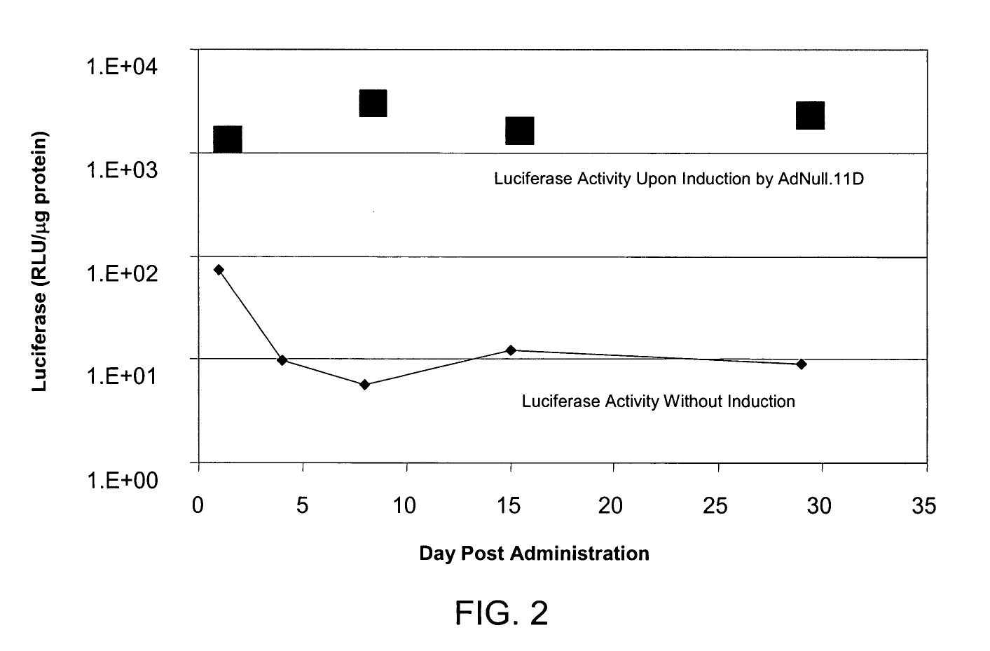

To activate expression of the luciferase gene in AdL.11D, 2×10⁸ pu of AdNull.11D, an isogenic vector that expressed no transgene, was administered to the eye via intravitreal injection. The AdNull.11D vector was either co-administered with or administered at 7, 14, or 28 days post-administration of 1×10⁷ pu of AdL.11D. The results of such an experiment are shown in FIG. 2. Expression from the AdL.11D vector absent induction by AdNull.11D declined over the first two weeks of the experiment. The addition of AdNull.11D enhanced expression at all time points tested for the duration of the 28 day experiment. The expression levels induced by administration of AdNull.11D were at least 10-fold higher than the peak expression levels obtained with AdL.11D alone on day 1. Maximum induction of about 100-fold was observed at day 14 and 28 post-administration when expression from AdL.11D was at its lowest levels.

The ability of AdNull.11D to activate expression was not restricted to adenoviral vectors containing the CMV promoter. An isogenic vector to AdL.11D having wild-type E4 sequences was constructed wherein the CMV promoter was replaced with an EF1α cellular promoter to generate AdEF.L. A dose of 1×10⁷ pu of either AdL.11D or AdEF.L was administered via intravitreal injection to the eye. A dose of 2×10⁸ pu of AdNull.11D was co-administered. Eyes were harvested at one day post-administration and levels of luciferase activity were determined. Expression of the luciferase gene from AdL.11D was stimulated approximately 10-fold by co-administration of AdNull.11D compared to expression in the absence of AdNull.11D. The same 10-fold induction of transcription also was observed for AdEF.L when co-administered with AdNull.11D.

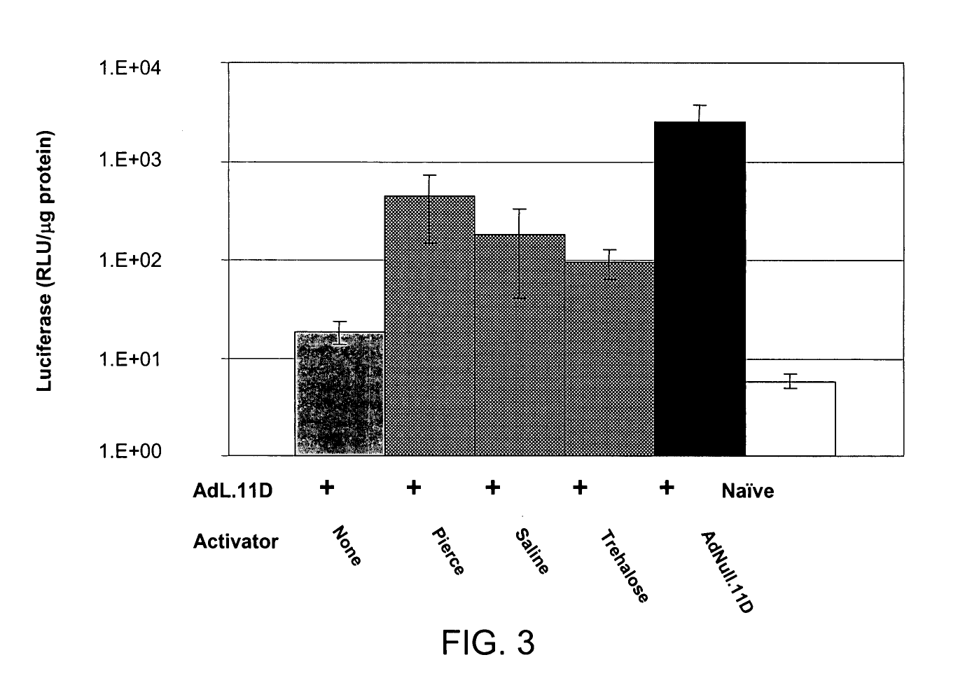

Induction of stress in the eye was found to be one component in the mechanism of expression activation. A total of 1×10⁷ pu of AdL.11D was delivered to the eye via intravitreal injection, followed by injections of either AdNull.11D, vector dilution buffer, or saline three days later. Alternatively, on the third day the eye was simply pierced with out delivering any material. The eyes were harvested on the fourth day and levels of luciferase expression determined (FIG. 3). The positive control of induction with AdNull.11D induced expression on the order of about 100-fold compared to the level of expression of AdL.11D in the absence of administration of the null vector. All three of the other treatments, administration of buffer or saline or piercing the eye, also induced expression. Simply piercing the eye yielded a 20-fold enhancement of expression.

The data provided by this example demonstrates super-activation of expression at any timepoint following transduction of a host cell of an adenoviral vector comprising a transgene by inducing a stress response in the host cell. Expression of the transgene was enhanced by inducing a stress response concurrently with vector administration, and expression was re-activated at all subsequent timepoints tested. Expression levels were enhanced to levels 10-fold higher than the highest (peak) level of expression obtained in the non-activated controls. Expression levels were re-activated to levels as high as 100-fold greater than the non-activated control at the same timepoints. In addition, administration of a null vector activated expression from adenoviral vectors regardless of promoter used to drive transgene expression, which demonstrates that the residual genomes remain completely expression competent. The addition of viral particles most likely further enhances the stress signal.

### Example 8

This example details the expression profiles of the UbC, JEM-1, and YY1 promoters in an adenoviral vector following intravitreal administration to the eye.

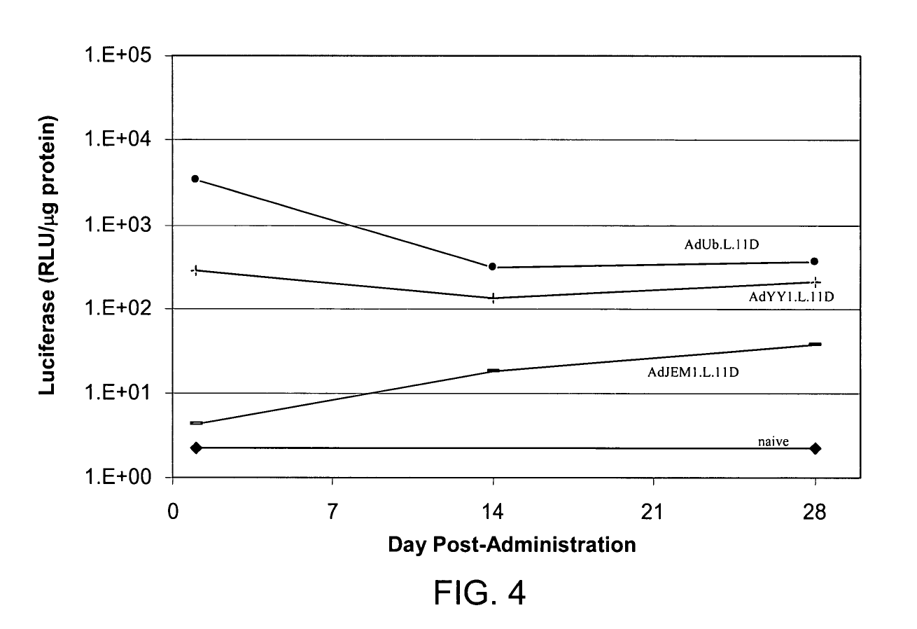

An adenoviral vector deficient in one or more essential gene functions of the E1, E3, and E4 regions of the adenoviral genome and comprising a luciferase gene (AdL.11D) is described in Examples 6 and 7. The adenoviral constructs were prepared wherein the CMV promoter of AdL.11D was replaced with the UbC promoter (AdUb.L.11D), the JEM-1 promoter (AdJEM1.L.11D), or the YY1 promoter (AdYY1.L.11D). A dose of 2×10⁸ pu of each adenoviral vector was injected intravitreally into the eyes of CD-1 nude mice. Ocular cells were isolated at various timepoints post-administration of the adenoviral vectors and luciferase activity was assayed, thereby providing a means of comparing expression levels over time. The expression profiles of the UbC, JEM-1, and YY1 promoters are illustrated in FIG. 4. Expression mediated by all of the promoters was steady over at least 28 days. Expression mediated by the UbC and YY1 promoters at 28 days post-administration was diminished no more than about 10-fold compared to peak expression levels. Expression mediated by the JEM-1 promoter steadily increased over 28 days.

### Example 9

This example demonstrates the upregulation of transgene expression in the eye by a drug.

An adenoviral vector was constructed as described herein. The adenoviral vector genome was deficient in one or more essential gene functions of the E1, E3, and E4 regions of the adenoviral genome and comprised a nucleic acid sequence encoding green fluorescence protein (GFP) (AdGFP.11D). The GFP gene under the control of the CMV immediate early promoter replaced the E1 region of the adenoviral genome while the E4 region was replaced with a spacer sequence that is not transcribed.

A total of 2×10⁸ particle units (pu) were injected intravitreously into C57BL/6 mice. On day 55 post-administration of the dose of adenoviral vector, retinoic acid was injected into the thigh muscle. An initial burst of expression was observed on day 1 post-administration of the adenoviral vector. Transgene expression waned to undetectable levels by day 55. On day 56 post-administration of the adenoviral vector (i.e., one day after administration of retinoic acid), GFP activity was detected, thereby indicating a re-activation of transgene expression.

In another experiment, 1×10⁷ particle units (pu) of an E1, E3, E4-deficient adenoviral vector comprising the luciferase gene (AdL.11D) were injected intravitreously into C57BL/6 mice. On day 7 post-administration of adenoviral vector, retinoic acid (100 μl, 50 mM) was systemically administered to the mice. Eyes were harvested on days 1, 7, and 8 to detect transgene expression. Initial transgene expression declined approximately 10-fold from day 1 to day 7. The administration of retinoic acid resulted in restoration of expression to peak levels, as measured by gene product activity. In other words, retinoic acid prompted restoration of transgene expression to levels similar to that detected on day 1 post-administration of adenoviral vector, resulting in a 10-fold activation of transcription.

This example demonstrated that transgene transcription can be upregulated by systemic administration of a drug at least 55 days following administration of an adenoviral vector encoding the transgene. In addition, transcription can be re-activated to peak levels.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of delivering a gene product to an eye, wherein the method comprises (a) administering to an eye of an animal a first expression vector comprising a nucleic acid sequence operably linked to a promoter and encoding a gene product, such that the expression vector transduces at least one ocular cell and the nucleic acid sequence is transcribed to produce a gene product, and (b) subsequently upregulating transcription of the nucleic acid sequence in the ocular cell by exposing the ocular cell to an exogenous material selected from the group consisting of saline, trehalose, a lipid, diclofenac sodium and misoprostol, dixlurenac, combretastatin, a protein kinase C (PKC) inhibitor, a tyrosine kinase inhibitor, a second expression vector not comprising the nucleic acid sequence, a histone deacetylase inhibitor, estrogen, a glucocorticoid, a glucocorticoid analog, retinoic acid, a retinoic acid analog, microwaves, ultrasound, a disaccharide, an aptamer, an siRNA, an immunosuppressant, a Cox-I inhibitor, a Cox-II inhibitor, an anti-inflammatory, aspirin, a prostaglandin analogue, a beta-blocker, hyaluronidase, pegaptanib sodium, tetrahydrozoline hydrochloride, an enzyme, an antibody, pigment epithelium-derived factor (PEDF), and dorzolamide hydrochloride.

2. The method of claim 1, wherein the first expression vector is an adenoviral vector.

3. The method of claim 2, wherein the adenoviral vector is replication-deficient.

4. The method of claim 3, wherein the adenoviral vector is deficient in at least one replication-essential gene function of the E1 region and/or the E4 region of the adenoviral genome of the adenoviral vector.

5. The method of claim 4, wherein all or part of the E1 and/or E4 region of the adenoviral genome of the adenoviral vector is removed.

6. The method of claim 1, wherein transcription is upregulated two or more times.

7. The method of claim 1, wherein transcription is upregulated at least once within one day of administering the first expression vector or adenoviral vector.

8. The method of claim 1, wherein the level of transcription of the nucleic acid sequence is at least 2-fold greater than the level of transcription of the nucleic acid sequence absent the upregulation of transcription.

9. The method of claim 1, wherein the level of transcription of the nucleic acid sequence at one day post-upregulating transcription is at least 20% the level of transcription of the nucleic acid sequence one day post-administration of the first expression vector or adenoviral vector.

10. The method of claim 1, wherein the second expression vector is an adenoviral vector.

11. The method of claim 10, wherein the adenoviral vector is deficient in all replication-essential gene functions of the E4 region of the adenoviral genome.

12. The method of claim 1, wherein the time between steps (a) and (b) is at least one day.

13. The method of claim 1, wherein the expression vector or adenoviral vector comprises a nucleic acid sequence encoding an inhibitor of angiogenesis and a nucleic acid sequence encoding a neurotrophic agent.

14. The method of claim 13, wherein the nucleic acid sequence encoding the inhibitor of angiogenesis and the nucleic acid sequence encoding the neurotrophic agent are the same nucleic acid sequence.

15. A method of prophylactically or therapeutically treating an animal for an ocular-related disorder, wherein the method comprises (a) administering to the animal a first expression vector comprising a nucleic acid sequence encoding an inhibitor of angiogenesis and/or a neurotrophic agent such that the expression vector transduces at least one ocular cell and the nucleic acid sequence is transcribed, and (b) subsequently upregulating transcription of the nucleic acid sequence by exposing the ocular cell to an exogenous material selected from the group consisting of saline, trehalose, a lipid, diclofenac sodium and misoprostol, dixlurenac, combretastatin, a protein kinase C (PKC) inhibitor, a tyrosine kinase inhibitor, a second expression vector not comprising the nucleic acid sequence, a histone deacetylase inhibitor, estrogen, a glucocorticoid, a glucocorticoid analog, retinoic acid, a retinoic acid analog, microwaves, ultrasound, a disaccharide, an aptamer, an siRNA, an immunosuppressant, a Cox-I inhibitor, a Cox-II inhibitor, an anti-inflammatory, aspirin, a prostaglandin analogue, a beta-blocker, hyaluronidase, pegaptanib sodium, tetrahydrozoline hydrochloride, an enzyme, an antibody, pigment epithelium-derived factor (PEDF), and dorzolamide hydrochloride, thereby upregulating expression of the inhibitor of angiogenesis and/or a neurotrophic agent to prophylactically or therapeutically treat the animal for an ocular-related disorder.

16. The method of claim 15, wherein the first expression vector is an adenoviral vector.

17. The method of claim 16, wherein the adenoviral vector is replication-deficient.

18. The method of claim 17, wherein the adenoviral vector is deficient in at least one replication-essential gene function of the E1 region and/or the E4 region of the adenoviral genome of the adenoviral vector.

19. The method of claim 18, wherein all or part of the E1 and/or E4 region of the adenoviral genome of the adenoviral vector is removed.

20. The method of claim 15, wherein transcription is upregulated two or more times.

21. The method of claim 15, wherein transcription is upregulated at least once within one day of administering the first expression vector or adenoviral vector.

22. The method of claim 15, wherein the level of transcription of the nucleic acid sequence is at least 2-fold greater than the level of transcription of the nucleic acid sequence absent the upregulation of transcription.

23. The method of claim 15, wherein the level of transcription of the nucleic acid sequence at one day post-upregulating transcription is at least 20% the level of transcription of the nucleic acid sequence one day post-administration of the first expression vector or adenoviral vector.

24. The method of claim 15, wherein the second expression vector is an adenoviral vector.

25. The method of claim 24, wherein the adenoviral vector is deficient in all replication-essential gene functions of the E4 region of the adenoviral genome.

26. The method of claim 15, wherein the time between steps (a) and (b) is at least one day.

27. The method of claim 15, wherein the expression vector or adenoviral vector comprises a nucleic acid sequence encoding an inhibitor of angiogenesis and a nucleic acid sequence encoding a neurotrophic agent.

28. The method of claim 27, wherein the nucleic acid sequence encoding the inhibitor of angiogenesis and the nucleic acid sequence encoding the neurotrophic agent are the same nucleic acid sequence.

29. The method of claim 15, wherein the ocular-related disorder is selected from the group consisting of ocular neovascularization, age-related macular degeneration, retinal tumors, diabetic retinopathy, macular edema, glaucoma, a retinal degenerative disease.

30. A method of delivering a gene product to a mammal, wherein the method comprises
(a) administering to a mammal
(i) an expression vector comprising a first nucleic acid sequence operably linked to a promoter, such that the expression vector transduces a host cell and the first nucleic acid sequence is transcribed to produce a gene product, and
(ii) a second nucleic acid sequence operably linked to a promoter and encoding a retinoic acid receptor, such that the second nucleic acid sequence is transcribed in the host cell to produce the retinoic acid receptor, and
(b) subsequently administering to the mammal a retinoic acid, thereby upregulating transcription of the first nucleic acid sequence in the host cell.

31. The method of claim 30, wherein the first expression vector is an adenoviral vector.

32. The method of claim 31, wherein the adenoviral vector is replication-deficient.

33. A method of delivering a gene product to a mammal, wherein the method comprises (a) administering to the mammal an adenoviral vector deficient in all replication-essential gene functions of the E4 region of the adenoviral genome and comprising a nucleic acid sequence operably linked to a promoter and encoding a gene product, such that the adenoviral vector transduces a host cell and the nucleic acid sequence is transcribed to produce a gene product, and (b) subsequently upregulating transcription of the nucleic acid sequence in the host cell by exposing the host cell to an exogenous material selected from the group consisting of saline, trehalose, a lipid, diclofenac sodium and misoprostol, dixlurenac, combretastatin, a protein kinase C (PKC) inhibitor, a tyrosine kinase inhibitor, a second expression vector not comprising the nucleic acid sequence, a histone deacetylase inhibitor, estrogen, a glucocorticoid, a glucocorticoid analog, retinoic acid, a retinoic acid analog, microwaves, ultrasound, a disaccharide, an aptamer, an siRNA, an immunosuppressant, a Cox-I inhibitor, a Cox-II inhibitor, an anti-inflammatory, aspirin, a prostaglandin analogue, a beta-blocker, hyaluronidase, pegaptanib sodium, tetrahydrozoline hydrochloride, an enzyme, an antibody, pigment epithelium-derived factor (PEDF), and dorzolamide hydrochloride.

34. The method of claim 33, wherein the adenoviral vector is deficient in at least one replication-essential gene function of the E1 region of the adenoviral genome of the adenoviral vector.

35. The method of claim 34, wherein all or part of the E1 and/or E4 region of the adenoviral genome of the adenoviral vector is removed.

36. The method of claim 33, wherein transcription is upregulated two or more times.

37. The method of claim 33, wherein transcription is upregulated at least once within one day of administering the first expression vector or adenoviral vector.

38. The method of claim 33, wherein the level of transcription of the nucleic acid sequence is at least 2-fold greater than the level of transcription of the nucleic acid sequence absent the upregulation of transcription.

39. The method of claim 33, wherein the level of transcription of the nucleic acid sequence at one day post-upregulating transcription is at least 20% the level of transcription of the nucleic acid sequence one day post-administration of the first expression vector or adenoviral vector.

40. The method of claim 33, wherein the second expression vector is an adenoviral vector.

41. The method of claim 40, wherein the adenoviral vector is deficient in all replication-essential gene finctions of the E4 region of the adenoviral genome.

42. The method of claim 33, wherein the time between steps (a) and (b) is at least one day.

43. The method of claim 33, wherein the expression vector or adenoviral vector comprises a nucleic acid sequence encoding an inhibitor of angiogenesis and a nucleic acid sequence encoding a neurotrophic agent.

44. The method of claim 43, wherein the nucleic acid sequence encoding the inhibitor of angiogenesis and the nucleic acid sequence encoding the neurotrophic agent are the same nucleic acid sequence.

45. A method of prophylactically or therapeutically treating an animal for an ocular-related disorder, wherein the method comprises contacting an ocular cell with an adenoviral vector comprising a nucleic acid sequence operably linked to a cellular promoter selected from the group consisting of a Ubiquitin C (UbC) promoter, a JEM-1 promoter, and a Ying Yang 1 (YY1) promoter, and encoding an inhibitor of angiogenesis and/or a neurotrophic agent, thereby resulting in the production of the inhibitor of angiogenesis and/or the neurotrophic agent to prophylactically or therapeutically treat the animal for an ocular-related disorder, with the proviso that, if the adenoviral vector is administered to a mouse at a dose of 2×10⁸ particles, the level of transcription of the nucleic acid sequence is not diminished more than three-fold at 28 days post-administration of the adenoviral vector compared to the level of transcription of the nucleic acid sequence at one day post-administration of the adenoviral vector.

46. The method of claim 45, wherein the adenoviral vector comprises a nucleic acid sequence encoding an inhibitor of angiogenesis and a nucleic acid sequence encoding a neurotrophic agent.

47. The method of claim 45, wherein the nucleic acid sequence encoding the inhibitor of angiogenesis and the nucleic acid sequence encoding the neurotrophic agent are the same nucleic acid sequence.

48. The method of claim 45, wherein the ocular-related disorder is ocular neovascularization, age-related macular degeneration, macular edema, glaucoma, diabetic retinopathy, retinal tumors, or a retinal degenerative disease.

49. The method of claim 45, wherein the adenoviral vector is replication-deficient.

50. The method of claim 45, wherein, if the adenoviral vector is administered to a mouse at a dose of 2×10⁸ particles, the level of transcription of the nucleic acid sequence is not diminished more than 5-fold at 28 days post-administration of the expression vector compared to the level of transcription of the nucleic acid sequence at one day post-administration.


SEQUENCE LISTING (1 sequence)

SEQ ID NO: 1 - DNA, 33014 bp, Artificial
  Synthetic construct
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggcccgggatcggtgatcaccgatccagacatgataagatacattgatgagtttggacaaaccacaactagaatgcagtgaaaaaaatgctttatttgtgaaatttgtgatgctattgctttatttgtaaccattataagctgcaataaacaagttcccggatctttctagctagtctagactagctagactcgagagcggccgcaatcgataagcttaggggcccctggggtccagaatcttgccaatgaagagaagggcccctgtgtctgtgtccctcagtacgaagatgaaaggctggttaaggtgatagtccagcgggaaggtgaggtgggcaggctgcagccctgggctgggggtggttcccgccccatcctcgttccactcaaagccagcccggtgttccacctgagtcagcttgatgggtttgcctgtgatcttgctaaagtctggtgaatcaaacaaggattgcagcttcatctcctgcagggacttggtgacttcgccttcgtaactcagcttcagcttggggacagtgaggaccgcctgcacggtcttcagttctcggtctatgtcatgaatgaactcggaggtgaggctctcctctatcaaggtcaaattctgggtcactttcaggggcaggaagaagatgatactcatgcttccggtcaagggcagctgggcaatcttgcagctgagatctgaatccaagccatagcgtaaaacagccttagggtccgacatcatggggaccctcacggtcctctcttcatccaagtagaaatcctcgagggaagtctttctggagtcaaactttgttacccactgccccttgaagtgcgccacaccgagaaggagaatgctgatctcatcgggaatttcctttgtggacctggcgagcttccctttcatctgcgcctgcacccagttgttgatctcttgcaggtccaagcgagggttgcccgtcaggactctgggcctggtcccatatgacttttccagaggtgccacaaagctggattttatgcgcagcttcttctcaaagacgatccgggaggcactcttgaggttcttctggggggcagtgaccgtgtcaaggagctccttataggtaccatggatgtctgggctgctgatcaagtcatagtagagagcccggtgaatgatggattctgttcgctgctccgctcccagcgagagggccgagagggccgtggccacactgagaggagacaggagcacgttggtcgtggggctcatgctggatcgcacccggtacaggtcatagccgaagttggagacagccgctgccagcttgttcacggggactttgaagaaaggatcctcctcctccaccagcgcccctgtgctgtcggggtctggggagccctcctccggggggctggcagggttctggcagctgctgtgcccgaggagggctccaatgcagaggagtagcaccagggcctgcatggtggaagcttgatatcgaattctgcagtgatcagggatcccagatccgtatagtgagtcgtattaggtaccggctgcagttggacctgggagtggacacctgtggagagaaaggcaaagtggatgtcattgtcactcaagtgtatggccagatctcaagcctgccacacctcaagtgaagccaagggggtgggcctatagactctataggcggtacttacgtcactcttggcacggggaatccgcgttccaatgcaccgttcccggccgcggaggctggatcggtcccggtgtcttctatggaggtcaaaacagcgtggatggcgtctccaggcgatctgacggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccggctgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgaacctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagcgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgccgttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcccgggatcggtgatcaccgatccagacatgataagatacattgatgagtttggacaaaccacaactagaatgcagtgaaaaaaatgctttatttgtgaaatttgtgatgctattgctttatttgtaaccattataagctgcaataaacaagttcccggatcgcgatccggcccgaggctgtagccgacgatggtgcgccaggagagttgttgattcattgtttgcctccctgctgcggtttttcaccgaagttcatgccagtccagcgtttttgcagcagaaaagccgccgacttcggtttgcggtcgcgagtgaagatccctttcttgttaccgccaacgcgcaatatgccttgcgaggtcgcaaaatcggcgaaattccatacctgttcaccgacgacggcgctgacgcgatcaaagacgcggtgatacatatccagccatgcacactgatactcttcactccacatgtcggtgtacattgagtgcagcccggctaacgtatccacgccgtattcggtgatgataatcggctgatgcagtttctcctgccaggccagaagttctttttccagtaccttctctgccgtttccaaatcgccgctttggacataccatccgtaataacggttcaggcacagcacatcaaagagatcgctgatggtatcggtgtgagcgtcgcagaacattacattgacgcaggtgatcggacgcgtcgggtcgagtttacgcgttgcttccgccagtggcgcgaaatattcccgtgcaccttgcggacgggtatccggttcgttggcaatactccacatcaccacgcttgggtggtttttgtcacgcgctatcagctctttaatcgcctgtaagtgcgcttgctgagtttccccgttgactgcctcttcgctgtacagttctttcggcttgttgcccgcttcgaaaccaatgcctaaagagaggttaaagccgacagcagcagtttcatcaatcaccacgatgccatgttcatctgcccagtcgagcatctcttcagcgtaagggtaatgcgaggtacggtaggagttggccccaatccagtccattaatgcgtggtcgtgcaccatcagcacgttatcgaatcctttgccacgcaagtccgcatcttcatgacgaccaaagccagtaaagtagaacggtttgtggttaatcaggaactgttcgcccttcactgccactgaccggatgccgacgcgaagcgggtagatatcacactctgtctggcttttggctgtgacgcacagttcatagagataaccttcacccggttgccagaggtgcggattcaccacttgcaaagtcccgctagtgccttgtccagttgcaaccacctgttgatccgcatcacgcagttcaacgctgacatcaccattggccaccacctgccagtcaacagacgcgtggttacagtcttgcgcgacatgcgtcaccacggtgatatcgtccacccaggtgttcggcgtggtgtagagcattacgctgcgatggattccggcatagttaaagaaatcatggaagtaagactgctttttcttgccgttttcgtcggtaatcaccattcccggcgggatagtctgccagttcagttcgttgttcacacaaacggtgatacgtacacttttcccggcaataacatacggcgtgacatcggcttcaaatggcgtatagccgccctgatgctccatcacttcctgattattgacccacactttgccgtaatgagtgaccgcatcgaaacgcagcacgatacgctggcctgcccaacctttcggtataaagacttcgcgctgataccagacgttgcccgcataattacgaatatctgcatcggcgaactgatcgttaaaactgcctggcacagcaattgcccggctttcttgtaacgcgctttcccaccaacgctgatcaattccacagttttcgcgatccagactgaatgcccacaggccgtcgagttttttgatttcacgggttggggtttctacaggacggaccatgcgttcgacctttctcttcttttttgggcccatgatggcagatccgtatagtgagtcgtattagctggttctttccgcctcagaagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctctagatgcatgctcgagcggccgccagtgtgatggatatctgcagaattccagcacactggcggccgttactagtggatccgagctcggtacccggccgttataacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatg